(19) United States
(12) Patent Application Publication
(10) Pub. No.: US 2024/0384438 A1
(43) Pub. Date: Nov. 21, 2024

(54) ANTIBODY AFFINITY MATURATION USING NATURAL LIABILITY-FREE CDRS

(71) Applicant: Rule-Based Medicine Inc, Santa Fe, NM (US)

(72) Inventors: Andrew Raymon Morton Bradbury, Santa Fe, NM (US); Sara D’Angelo, Santa Fe, NM (US); Andre A. Teixeira, Santa Fe, NM (US)

(73) Assignee: Rules-Based Medicine Inc., Austin, TX (US)

(21) Appl. No.: 18/577,398

(22) PCT Filed: Jul. 7, 2022
(86) PCT No.: PCT/US2022/036422

§ 371 Date: Jan. 8, 2024

Related U.S. Application Data
(60) Provisional application No. 63/218,919, filed on Jul. 7, 2021.

(51) Int. Cl. C40B 40/10; C07K 16/00; C40B 50/00

(52) U.S. Cl. CPC C40B 40/10; C07K 16/005; C40B 50/00; C07K 2317/565

(57) ABSTRACT

Provided herein is an improved method of antibody affinity maturation that uses true natural CDRs from a population of naturally occurring antibodies targeting a single antigen or antigenic epitope such that the improved method produces functional antibodies having low-picomolar affinity antibodies. Also provided herein is an antibody library where the CDRs within a single antibody member of the library are a combination of CDR sequences of naturally occurring antibodies and one or more CDRs are derived from different naturally occurring antibodies targeting a single antigen or antigenic epitope.

### CROSS-REFERENCE TO RELATED APPLICATIONS

This application claims the benefit under 35 U.S.C. § 119(e) of U.S. Provisional Application No. 63/218,919, filed Jul. 7, 2021, the contents which are incorporated by reference herein in their entirety.

### FIELD OF THE DISCLOSURE

This disclosure relates to antibody affinity maturation of antibodies targeting an antigen or antigenic epitope using natural liability-free complementarity-determining regions (CDRs).

### BACKGROUND

The quality of a monoclonal antibody is often measured by its affinity to the target antigen. Hence, a common step in the development of a therapeutic antibody is submitting selected leads to affinity maturation campaigns on the assumption that higher affinities will lead to higher drug potency.

Campaigns follow three basic steps: (1) diversification, (2) selection, and (3) validation, and many successful methods have been used. Diversification has been achieved by techniques such as error-prone PCR (Gram, Marconi et al. 1992, Daugherty, Chen et al. 2000), chain shuffling (Marks, Griffiths et al. 1992, Park, Lee et al. 2000, Lou, Geren et al. 2010), targeted CDR mutation (Yang, Green et al. 1995, Schier, Bye et al. 1996, Schier and Marks 1996, Rajpal, Beyaz et al. 2005, Barderas, Desmet et al. 2008, Tiller, Chowdhury et al. 2017), and others (Low, Holliger et al. 1996, Boder, Midelfort et al. 2000), and will usually create some variants with higher affinities, and many with unchanged or lower affinities. By using display platforms, such as phage (Yang, Green et al. 1995, Schier and Marks 1996, Schier, McCall et al. 1996, Lamdan, Gavilondo et al. 2013), yeast (Boder, Midelfort et al. 2000, Tiller, Chowdhury et al. 2017), or ribosome display (Hanes, Jermutus et al. 1998, Hanes, Schaffitzel et al. 2000, Chan, Jenkinson et al. 2020) and the application of selective pressure, the new variants with improved affinities can be separated from the rest. Finally, the produced population can either undergo additional rounds of diversification and selection, or individual clones validated for affinity.

While these diversification techniques are efficient in creating molecules with improved affinities, they often create challenges for downstream development. For example, a biologically validated lead undergoing maturation must retain the same epitope: aggressive randomization strategies such as chain shuffling may lead to epitope drift (Ohlin, Owman et al. 1996) and instead of increased potency may result in a loss of biological activity. Random mutations throughout the entire V_{H} or V_{L} domains will inevitably mutate regions unrelated to antigen binding and may generate unstable molecules with poor developability (Julian, Lee et al. 2015). The same is true for the insertion of degenerate sequences within CDRs that can lead to unstable antibodies arising from unnatural sequences causing covariance violations which may affect overall antibody structure (Chou, Némethy et al. 1989, Du, Wei et al. 2003).

Moreover, none of these techniques addresses the issue of sequence liabilities such as glycosylation, asparagine deamidation, aspartate isomerization, aggregation prone motifs, and others, which can negatively impact drug development.

### SUMMARY

The present disclosure is based, at least in part, on an improved method of antibody affinity maturation using natural complementarity-determining regions (CDRs) that are substantially free of liabilities. The CDRs are natural, and derived from sequencing rearranged antibody genes. These CDRs are further screened for member sequence liabilities that potentially negatively impact developability of the assembled antibody from the expressed light chain and the expressed heavy chain carrying these CDRs. The CDRs having these liabilities are removed from consideration in assembling the antibody libraries contemplated herein. The remaining natural, liability-free CDRs are combined and assembled into antibodies and screened for improved antigen-binding affinity compared to the control, parental antibody, improvement affinity can range ˜picomolar binding affinity.

Accordingly, one aspect of the present disclosure provides an antibody library comprising a plurality of antibodies, wherein each antibody of the plurality of antibodies comprises the following CDR sequences: a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence, wherein two or more of the CDR sequences are the same for each of the plurality of antibodies (“invariant CDR sequences”); and wherein each of the remaining CDR sequences comprise a plurality of unique CDRs derived from sequences of the CDRs found in naturally occurring antibodies (“variant CDR sequences”). “Naturally occurring antibodies” means antibodies that do not reflect antibody engineering or other genetic engineering.

In some embodiments, the two or more invariant CDR sequences may be selected from the group consisting of: (i) V_{L}-CDR3, V_{H}-CDR1, V_{H}-CDR2, and V_{H}-CDR3; (ii) V_{L}-CDR1, V_{L}-CDR2, V_{H}-CDR1, V_{H}-CDR2, and V_{H}-CDR3; (iii) V_{L}-CDR1, V_{L}-CDR2, V_{L}-CDR3, and V_{H}-CDR3; (iv) V_{L}-CDR1, V_{L}-CDR2, and V_{H}-CDR3; and (v) V_{L}-CDR1, V_{L}-CDR2, V_{L}-CDR3, V_{H}-CDR1 and V_{H}-CDR2.

In some embodiments, the invariant CDR sequences may be derived from a parental antibody binding to a single antigen or antigenic epitope.

Also provided in the present disclosure is an antibody library comprising a plurality of antibodies, wherein each antibody of the plurality of antibodies comprises the following CDR sequences: a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence, wherein two or more of the CDR sequences are the same for each antibody of the plurality of antibodies (“invariant CDR sequences”); and wherein each of the remaining CDR sequences (“variant CDR sequences”), except the HCDR3, comprises a plurality of CDRs derived from the sequences of CDRs found in naturally occurring antibodies, and the HCDR3 comprises a plurality of variants of the parental HCDR3. In some embodiments, the HCDR3 comprises single mutations of the parental HCDR3.

In some embodiments, the two or more invariant CDR sequences may be selected from the group consisting of: (i) V_{L}-CDR3, V_{H}-CDR1, V_{H}-CDR2, and V_{H}-CDR3; (ii) V_{L}-CDR1, V_{L}-CDR2, V_{H}-CDR1, V_{H}-CDR2, and V_{H}-CDR3; (iii) V_{L}-CDR1, V_{L}-CDR2, V_{L}-CDR3, and V_{H}-CDR3; and (iv) V_{L}-CDR1, V_{L}-CDR2, V_{L}-CDR3, V_{H}-CDR1, and V_{H}-CDR2.

In some embodiments, the invariant CDR sequences may be derived from a parental antibody binding to a single antigen or antigenic epitope.

In any of the antibody libraries disclosed above and herein, the invariant CDR sequences may comprise a combination of V_{H}-CDR1, V_{H}-CDR2, V_{H}-CDR3, V_{L}-CDR1, V_{L}-CDR2 and/or V_{L}-CDR3. In some embodiments, the invariant and variant CDR sequences are free of one or more of the following liabilities: (i) a glycosylation site, (ii) a deamidation site, (iii) an isomerization site, (iv) unpaired cysteine, (v) net charge greater than 1, (vi) a tripeptide motif containing at least two aromatic residues, (vii) a motif that promotes aggregation, (viii) a poly specificity site, (ix) a protease sensitive site, (x) an integrin binding site, (xi) a lysine glycosylation site, (xii) a metal catalyzed fragmentation site, (xiii) a poly specificity aggregation site, and (xiv) a streptavidin binding motif.

In some embodiments, the glycosylation site may comprise the motif NXS, NXT, or NXC, in which X represents any naturally-occurring amino acid residue except for proline.

In some embodiments, the deamidation site may comprise the motif of NG, NS, NT, NN, NA, NH, ND, GNF, GNY, GNT, or GNG.

In some embodiments, the isomerization site may comprise the motif of DT, DH, DS, DG, or DD.

In some embodiments, the tripeptide may be HYF or HWH.

In some embodiments, the motif that promotes aggregation may comprise the motif of FHW.

In some embodiments, the poly specificity site may comprise the motif of GG, GGG, RR, VG, W, WV, WW, WWW, YY, or WXW, in which X represents any amino acid residue.

In some embodiments, the protease cleavage site may comprise the motif of DX, in which X is P, G, S, V, Y, F, Q, K, L, or D.

In some embodiments, the integrin binding site may comprise RGD, RYD, LDV, or KGD.

In some embodiments, the lysine glycosylation site may comprise KE, EK, or ED.

In some embodiments, the metal catalyzed fragmentation site may comprise the motif of HS, SH, KT, HXS, or SXH, in which X represents any amino acid residue.

In some embodiments, the poly specificity aggregation site may comprise a motif of X1X2X3, wherein each of X1, X2, and X3 independently is selected from the group consisting of F, I, L, V, W and Y.

In some embodiments, the streptavidin binding motif may comprise the motif HPQ, EPDW, PWXWL, in which X represents any amino acid residue, GDWVFI, or PWPWLG.

In some embodiments, any one of the antibody libraries disclosed above and herein may be a full-length antibody library, a Fab antibody library, a single-chain antibody library, or a single domain antibody library.

In some embodiments, any one of the antibody libraries disclosed above and herein may be a human antibody library.

In another aspect, the present disclosure provides a method for generating an antibody library, the method comprising: (a) selecting two or more CDR sequences from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence; and (b) generating an antibody library comprising a plurality of antibodies, wherein each antibody of the plurality of antibodies comprises the following CDR sequences: (i) the two or more CDR sequences selected in (a) (“invariant CDR sequences”); and (ii) a unique combination of remaining CDR sequences not selected in (a) (“variant CDR sequences”), wherein the variant CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence and are derived from the sequences of CDRs found in naturally occurring antibodies.

In another aspect, the present disclosure also provides a method for generating an antibody library, the method comprising: (a) selecting two or more CDR sequences from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence; and (b) generating an antibody library comprising a plurality of antibodies, wherein each antibody of the plurality of antibodies comprises the following CDR sequences: (i) the two or more CDR sequences selected in (a) (“invariant CDR sequences”); and (ii) a unique combination of remaining CDR sequences not selected in (a) (“variant CDR sequences”), wherein the variant CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence and are derived from the sequences of CDRs found in naturally occurring antibodies for all CDR sequences except V_{H}-CDR3 (HCDR3), wherein the HCDR3 comprises a plurality of variants of the parental HCDR3. In some embodiments, the HCDR3 may comprise single mutation(s) of the parental HCDR3 sequence.

Still in another aspect, provided herein is a method for identifying an antibody with improved affinity to a target antigen or a target antigenic epitope, the method comprising: (a) generating an antibody library according to any of the method described above and herein, wherein the two or more invariant CDR sequences are derived from the parental antibody to be affinity matured; and (b) screening the antibody library to isolate an antibody that binds more tightly to the target antigen or target antigenic epitope relative to a control, thereby identifying an antibody with improved affinity.

Still in another aspect, provided herein is a method for improving the developability of an antibody that binds a target antigen or a target antigenic epitope, the method comprising: (a) generating an antibody library according to any of the methods described above and herein, wherein the two or more invariant CDR sequences are derived from the parental antibody to be affinity matured, and wherein the invariant and variant CDR sequences are free of one or more of sequence liabilities; and (b) screening the antibody library to isolate an antibody that binds more tightly to the target antigen or the target antigenic epitope relative to a control and lacks one or more of the sequence liabilities found in the CDRs of the original antibody, thereby improving the developability of the antibody.

In some embodiments, the two or more invariant CDR sequences may be selected from the group consisting of: (i) V_{L}-CDR3, V_{H}-CDR1, V_{H}-CDR2, and V_{H}-CDR3; (ii) V_{L}-CDR1, V_{L}-CDR2, V_{H}i-CDR1, V_{H}-CDR2, and V_{H}-CDR3; (iii) V_{L}-CDR1, V_{L}-CDR2, V_{L}-CDR3, and V_{H}-CDR3; (iv) V_{L}-CDR1, V_{L}-CDR2, and V_{H}-CDR3; and (v) V_{L}-CDR1, V_{L}-CDR2, V_{L}-CDR3, V_{H}-CDR1 and V_{H}-CDR2.

In some embodiments, the invariant and variant CDR sequences are free of one or more of the following liabilities: (i) a glycosylation site, (ii) a deamidation site, (iii) an isomerization site, (iv) unpaired cysteine, (v) net charge greater than 1, (vi) a tripeptide motif containing at least two aromatic residues, (vii) a motif that promotes aggregation, (viii) a poly specificity site, (ix) a protease sensitive site, (x) an integrin binding site, (xi) a lysine glycosylation site, (xii) a metal catalyzed fragmentation site, (xiii) a poly specificity aggregation site, and (xiv) a streptavidin binding motif.

In some embodiments, the glycosylation site may comprise the motif NXS, NXT, or NXC, in which X represents any naturally-occurring amino acid residue except for proline.

In some embodiments, the deamidation site may comprise the motif of NG, NS, NT, NN, NA, NH, ND, GNF, GNY, GNT, or GNG.

In some embodiments, the isomerization site may comprise the motif of DT, DH, DS, DG, or DD.

In some embodiments, the tripeptide may be HYF or HWH.

In some embodiments, the motif that promotes aggregation may comprise the motif of FHW.

In some embodiments, the poly specificity site may comprise the motif of GG, GGG, RR, VG, W, WV, WW, WWW, YY, or WXW, in which X represents any amino acid residue.

In some embodiments, the protease cleavage site may comprise the motif of DX, in which X is P, G, S, V, Y, F, Q, K, L, or D.

In some embodiments, the integrin binding site may comprise RGD, RYD, LDV, or KGD.

In some embodiments, the lysine glycosylation site may comprise KE, EK, or ED.

In some embodiments, the metal catalyzed fragmentation site may comprise the motif of HS, SH, KT, HXS, or SXH, in which X represents any amino acid residue.

In some embodiments, the poly specificity aggregation site may comprise a motif of X1X2X3, wherein each of X1, X2, and X3 independently is selected from the group consisting of F, I, L, V, W and Y.

In some embodiments, the streptavidin binding motif may comprise the motif HPQ, EPDW, PWXWL, in which X represents any amino acid residue, GDWVFI, or PWPWLG.

In some embodiments, any one of the antibody libraries disclosed above and herein may be a full-length antibody library, a Fab antibody library, a single-chain antibody library, or a single domain antibody library.

In some embodiments, any one of the antibody libraries disclosed above and herein may be a human antibody library.

Any of the antibody libraries disclosed herein may be of a suitable format, e.g., a library of full-length antibodies, a library of antigen-binding fragments such as Fab fragments, a library of single-chain antibodies, or a library of single-domain antibodies (e.g., VHH antibodies). In some examples, the antibody library disclosed herein may be a human antibody library. In other examples, the antibody library disclosed herein may be a camelid VHH antibody library.

In yet another aspect, the present disclosure provides a VHH library comprising a plurality of antibodies, wherein each antibody of the plurality of antibodies comprises the following CDR sequences: a CDR1 sequence, a CDR2 sequence, and a CDR3 sequence, wherein one or more of the CDR sequences is(are) the same for each antibody of the plurality of antibodies (invariant CDR Sequence(s)); and wherein each of the remaining CDRs comprises a plurality of CDRs derived from the sequences of CDRs found in naturally occurring antibodies (variant CDRs).

In some embodiments, the invariant CDR sequence(s) is(are) derived from a parental VHH binding to a single antigen or antigenic epitope.

In still yet another aspect, the present disclosure provides a VHH library comprising a plurality of antibodies, wherein each antibody of the plurality of antibodies comprises the following CDR sequences: a CDR1 sequence, a CDR2 sequence, and a CDR3 sequence, wherein one or more of the CDR sequences is(are) the same for each antibody of the plurality of antibodies (“invariant CDR Sequence(s)”); and wherein each of the remaining CDRs, except the HCDR3, comprises a plurality of CDRs derived from the sequences of CDRs found in naturally occurring antibodies (“variant CDRs”), and wherein the HCDR3 comprises a plurality of variants of the parental HCDR3. In some embodiments, the HCDR3 comprises single mutations of the parental HCDR3.

In some embodiments, the invariant CDR sequence(s) is(are) derived from a parental VHH binding to a single antigen or antigenic epitope.

In still yet another aspect, the present disclosure provides a method for generating a VHH library, the method comprising: (a) selecting one or more CDR sequences from the group consisting of a CDR1 sequence, a CDR2 sequence, and a CDR3 sequence; and (b) generating an antibody library comprising a plurality of antibodies, wherein each antibody of the plurality of antibodies comprises the following CDR sequences: (i) the one or more CDR sequences selected in (a) (“invariant CDR sequence(s)”); and (ii) a unique combination of remaining CDR sequences not selected in (a) (“variant CDR sequences”), wherein each of the variant CDR sequences comprises a plurality of CDRs derived from the sequences of CDRs found in naturally occurring antibodies.

In still yet another aspect, the present disclosure provides a method for generating a VHH library, the method comprising: (a) selecting one or more CDR sequences from the group consisting of a CDR1 sequence, a CDR2 sequence, and a CDR3 sequence; and (b) generating an antibody library comprising a plurality of antibodies, wherein each antibody of the plurality of antibodies comprises the following CDR sequences: (i) the one or more CDR sequences selected in (a) (“invariant CDR sequence(s)”); and (ii) a unique combination of remaining CDR sequences not selected in (a) (“variant CDR sequences”), wherein each of the variant CDR sequences, except the HCDR3, comprise a plurality of CDRs derived from the sequences of CDRs found in naturally occurring antibodies, and wherein the HCDR3 comprises a plurality of variants of the parental HCDR3. In some embodiments, the CDR3 may comprise single mutations of the parental HCDR3.

In some embodiments, the CDR sequences are free of one or more of the following sequence liabilities: (i) a glycosylation site, (ii) a deamidation site, (iii) an isomerization site, (iv) unpaired cysteine, (v) net charge greater than 1, (vi) a tripeptide motif containing at least two aromatic residues, (vii) a motif that promotes aggregation, (viii) a poly specificity site; (ix) a protease sensitive site, (x) an integrin binding site, (xi) a lysine glycosylation site, (xii) a metal catalyzed fragmentation site, (xiii) a poly specificity aggregation site; and (xiv) a streptavidin binding motif.

For example, the glycosylation site may comprise the motif NXS, NXT, or NXC, in which X represents any naturally-occurring amino acid residue except for proline; the deamidation site may comprise the motif of NG, NS, NT, NN, NA, NH, ND, GNF, GNY, GNT, or GNG; wherein the isomerization site may comprise the motif of DT, DH, DS, DG, or DD; tripeptide is HYF or HWH; the motif that promotes aggregation may comprise the motif of FHW; the polyspecificity site may comprise the motif GG, GGG, RR, VG, W, WV, WW, WWW, YY, or WXW, in which X represents any amino acid residue; the protease cleavage site may comprise the motif of DX, in which X is P, G, S, V, Y, F, Q, K, L, or D; the integrin binding site may comprise RGD, RYD, LDV, or KGD; the lysine glycosylation site comprises KE, EK, or ED; the metal catalyzed fragmentation site may comprise the motif of HS, SH, KT, HXS, or SXH, in which X represents any amino acid residue; the poly specificity aggregation site may comprise a motif of X1X2X3, wherein each of Xi, X2, and X3 independently is selected from the group consisting of F, I, L, V, W and Y; and the streptavidin binding motif may comprise the motif HPQ, EPDW, PWXWL, in which X represents any amino acid residue, GDWVFI, or PWPWLG.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A shows the schematic representation of the parental antibody scFv and the three libraries produced in phase 1: L1L2, L3, and H1H2. Each library has diversity introduced in the indicated CDRs. These phase 1 libraries allow for screening and selecting a pool of the best affinities for the respective tested CDRs, the pool of best affinity CDRs are then to be later used in the phase 2 for generating combinatorial libraries having at least three CDRs replaced and three natural non-replaced CDRs. FIG. 1B shows the sensorgram of the parental antibody binding to the antigen generated by surface-plasmon resonance in increasing antigen concentrations (0.16 nM to 100 nM with 5-fold increase in each step). FIG. 1C shows the schematic for creating a L1L2 library in phase 1; this is an exemplary for also creating a L3, and a HIH2 libraries. An L1 sub-library pool is generated by PCR using specific primers as described herein (the LCDR1 pool). Similarly, an L2 sub-library is generated by PCR (the LCDR2 pool). PCR with selected designed primers then allow the generation of the L1L2 library from these two sub-libraries and assembling L1L2 into the parental antibody scFv (which has unchanged LCDR3 and HCDRs) and thereby produced the L1L2 library in phase 1. Each library has diversity introduced in the indicated CDRs.

FIG. 2A shows the schematic representation of yeast display selections using equilibrium and kinetic protocols. FIG. 2B shows the outline of the selection rounds performed in phase 1 with the three phase 1 libraries (L1L2, L3, H1H2). FIG. 2C shows the yeast display binding profiles of the parental scFv and the three phase 1 libraries at increasing antigen concentration assessed by flow cytometry. Binding to antigen (APC fluorescence) is shown on the Y axis and scFv display (PE fluorescence) is shown on the X axis. FIG. 2D shows the yeast display binding profile of the parental scFv and the libraries after 5 rounds of selection. A gate representing the parental population is shown in all plots for comparison. In the last two columns, after labeled antigen incubation, cells were washed and incubated with unlabeled antigen for 2 h and 4 h to evaluate the stability of binding.

FIG. 3A shows the schematic representation of parental antibody scFv and the two Combo libraries produced: Combo 1 has diversity in LCDR1-3 and HCDR1-2 and Combo 2 has diversity in LCDR3 and HCDR1-2. FIG. 3B shows the outline of the selection rounds performed in phase 2 with the two Combo libraries. FIG. 3C shows the yeast display binding profile of the combo libraries before any rounds of selection were performed. Binding to antigen (APC fluorescence) is shown on the Y axis and scFv display (PE fluorescence) is shown on the X axis. FIG. 3D shows the yeast display binding profile of the parental scFv and the combo libraries after 3 rounds of selection.

FIG. 4A shows the Venn diagram representing the number of unique clones identified by Sanger sequencing coming from Combo libraries 1 and 2. FIG. 4B shows the chord diagram showing the connections between each of the CDRs identified. FIG. 4C shows the sequence logos comparing the CDRs from the parental scFv, designed for the library, and observed in the sequenced clones. Letter heights indicate the frequency of the given amino acid and letter width represent frequency of non-gap at the position. Dashed squared show regions that converged for an amino acid different from the parental. FIG. 4D shows the heat map showing the number of amino acid changes from parental in each CDR and the total number per clone. Each row represents an identified clone. Clones are ordered from least to most total changes. FIG. 4E shows the histograms representing the number of amino acid changes in each CDR identified and the total number of changes per clone. FIG. 4F is a histogram showing the total edit distance for each selected clone compared to the parental clone. The distance is calculated individually for each CDR and then summed to find the total distance

FIG. 5A shows the antigen binding to yeast displaying the parental scFv when competing with 23 affinity matured clones (A01 to B12) and an unrelated scFv-Fc (control). All values are shown normalized by the control. FIG. 5B shows the observed on—(ka) and off-rates (kd) for the parental and identified clones. Isoaffinity curves are shown as dashed diagonal lines. FIG. 5C shows the SPR sensorgrams for 23 of the identified clones shown in duplicate (side-by-side). Name of the clone and calculated KD is shown in each plot. Measurements where the calculated off-rate (kd) is estimated to be less than 10⁻⁵ s⁻¹ are outlined in black.

FIG. 6 shows the flow cytometric analysis of antigen binding to the yeast displaying the parental scFv when competing with 23 affinity matured clones (A01 to B12) and an unrelated scFv-Fc (control). The scFv-Fc supernatants were incubated with the labeled antigen (10 nM) for 15 minutes, then yeast cells displaying the parental molecule were added, followed by fluorescent staining to detect binding to the antigen.

FIG. 7A shows the schematic representation of the parental antibody scFv and the four libraries produced in phase 1: H1H2, H3, L1L2 and L3. Each library has diversity introduced in the indicated CDRs. These phase 1 libraries allow for screening and selecting a pool of the best affinities for the respective tested CDRs, the pool of best affinity CDRs are then to be later used in the phase 2 for generating combinatorial libraries having the six CDRs replaced. FIG. 7B shows the schematic for creating a H1H2 library in phase 1; this is an exemplary for also creating a H3, L1L2 and L3 libraries. An H1 sub-library pool is generated by PCR using specific primers as described herein (the HCDR1 pool). Similarly, an H2 sub-library is generated by PCR (the HCDR2 pool). PCR with selected designed primers then allow the generation of the H1H2 library from these two sub-libraries and assembling H1H2 into the parental antibody scFv (which has unchanged HCDR3 and LCDRs) and thereby produced the H1H2 library in phase 1. Each library has diversity introduced in the indicated CDRs.

FIG. 8A shows the yeast display binding profiles of the four phase 1 libraries at increasing antigen concentration assessed by flow cytometry. Binding to antigen (APC fluorescence) is shown on the Y axis and scFv display (PE fluorescence) is shown on the X axis. FIG. 8B shows the outline of the selection rounds performed in phase 1 with the four phase 1 libraries (H1H2, H3, L1L2 and L3). FIG. 8C shows the yeast display binding profile of the four phase 1 libraries (H1H2, H3, L1L2 and L3) after 3 rounds of selection.

FIG. 9A shows the schematic representation of parental antibody scFv and the Combo libraries produced: Combo 1 has diversity in LCDR1-3 and HCDR1-3, while the HCDR3 for Combo 2 remains constant. FIG. 9B shows the outline of the four selection rounds performed in phase 2 with the two Combo libraries. FIG. 9C shows the yeast display binding profile of the Combo libraries before any rounds of selection were performed. Binding to antigen (APC fluorescence) is shown on the Y axis and scFv display (PE fluorescence) is shown on the X axis. FIG. 9D shows the yeast display binding profile of the parental scFv and the combo libraries after four rounds of selection, at 1 nM and 1 μM of antigen. FIG. E compares the yeast display binding profile of the parental scFv and the combo libraries after four rounds of selection, when stained with 750 nM of antigen, and at different release time points.

FIG. 10A shows the Venn diagram representing the number of unique clones identified by Sanger sequencing from Combo libraries 1 and 2, derived from the four phase 1 libraries. FIG. 10B shows the heat map showing the number of amino acid changes from parental in each CDR and the total number per clone. Each row represents an identified clone. Clones are ordered from least to most total changes. FIG. 10C shows the histograms representing the number of amino acid changes in each CDR identified and the total number of changes per clone. FIG. 10D shows the sequence logos comparing the CDRs from the parental scFv, designed for the library, and observed in the sequenced clones. Letter heights indicate the frequency of the given amino acid and letter width represent frequency of non-gap at the position. Mutational frequencies of the HCDR3 were too low to be visualized in the sequence logo.

### DETAILED DESCRIPTION

Affinity maturation is a necessary step for the development of potent therapeutic molecules from a library of naïve antibodies targeting and binding an antigen or an antigenic epitope. The quality of a monoclonal antibody is often measured by its affinity to the target antigen or the target antigenic epitope. Hence, a common step in the development of a therapeutic antibody is submitting selected leads to affinity maturation campaigns on the assumption that higher affinities will lead to higher drug potency. Many techniques are used for antibody affinity maturation, including error-prone PCR, chain shuffling, targeted CDR mutation, and others. These are effective but can negatively affect antibody stability or alter epitope recognition. Moreover, they do not address the presence of sequence liabilities, such as glycosylation, asparagine deamidation, aspartate isomerization, aggregation motifs, and others that potentially could affect the developability of the lead antibody arising from the naïve library targeting an antigen. All these can potentially create the need for new rounds of engineering or even abolish the usefulness of the antibody as a therapeutic molecule.

The present disclosure provides improved methods of antibody affinity maturation using natural complementarity-determining regions (CDRs) that are substantially free of sequence liabilities as well as antibody libraries so produced. By virtue of being comprised of highly functional members, the antibody libraries of the present disclosure are functionally much larger than libraries of similar genetic size, in which antibodies are present that contain any of the sequence liabilities. In other words, the antibody libraries disclosed herein have a much larger effective diversity.

As used herein, the term “liability” refers to a motif in an antibody that would negatively affect one or more desired features of the antibody (e.g., stability, good expression in an expression or display system, proper folding, no or reduced aggregation, solubility, no or reduced integrin binding, no or reduced glycosylation, no or reduced deamidation, no or reduced isomerization, no unpaired cysteine, or no or reduced protease sensitivity, etc.).

The present disclosure demonstrated the possibility of performing affinity maturation of a low-nanomolar affinity antibody to the low-picomolar range by a) replacing all CDRs, except HCDR3, with a collection of known human CDRs; and b) also replacing the HCDR3 with limited variants. More specifically, provided herein is a method for generating a combinatorial antibody library with improved epitope-binding affinity, improvement in the order 103, by switching out one or more CDRs from a collection of known CDRs obtained from a naïve naturally occurring antibody library, and for limited variants in HCDR3.

Among all the antibodies binding the same target antigen, a single lead antibody from this naïve naturally occurring antibody library is selected as the parent antibody which would provide V_{H} and V_{L} scaffold framework for replacing the CDRs described below. That lead antibody of lower affinity has three V_{H}-CDRs and three V_{L}-CDRs as its “natural” or parental CDRs. For replacing CDRs in the V_{H} and/or V_{L}, the assortments of natural CDRs are mixed and assembled into a respective V_{H} or V_{L} such that the arrangements of all the V_{H}-CDRs1-3 and V_{L}-CDRs1-3 are not as they are in the naturally occurring antibody library, i.e., artificial combinations of the natural CDRs.

As used herein, the term “developability” encompasses the feasibility of molecules to successfully progress from discovery to development via evaluation of their physicochemical properties.

Additionally, the disclosure provides an antibody library comprising a plurality of antibodies, wherein each antibody therein comprises: a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence; wherein one or more CDR sequences selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence, are the same for each antibody of the plurality of antibodies; and each of the remaining non-selected CDRs making up the V_{H} and V_{L} is derived from the sequences of CDRs found in naturally occurring antibodies.

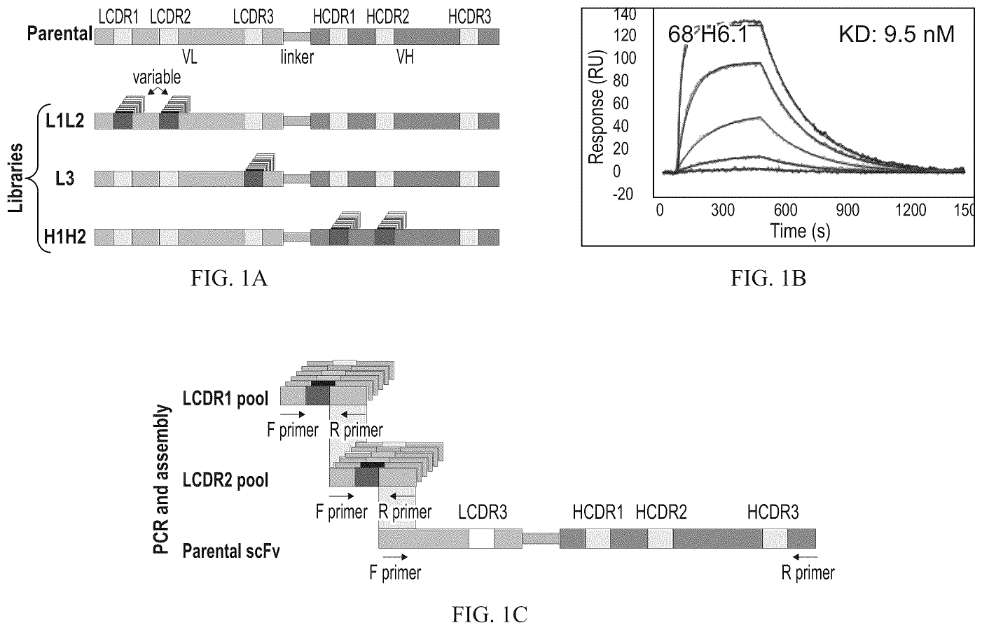

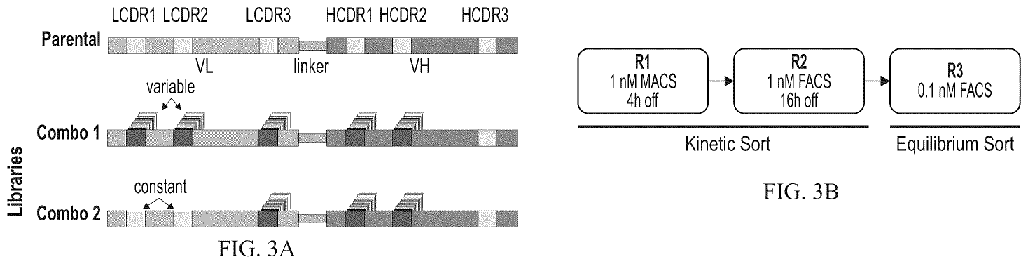

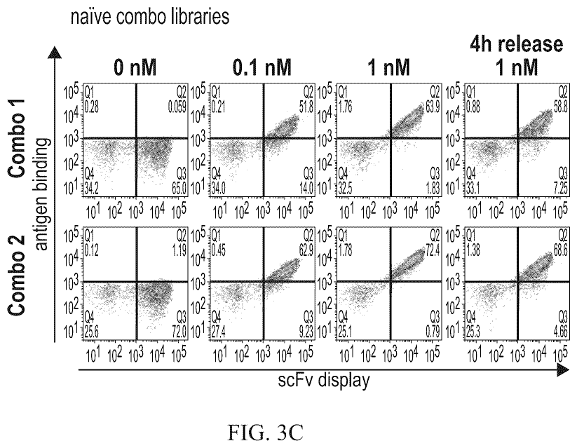

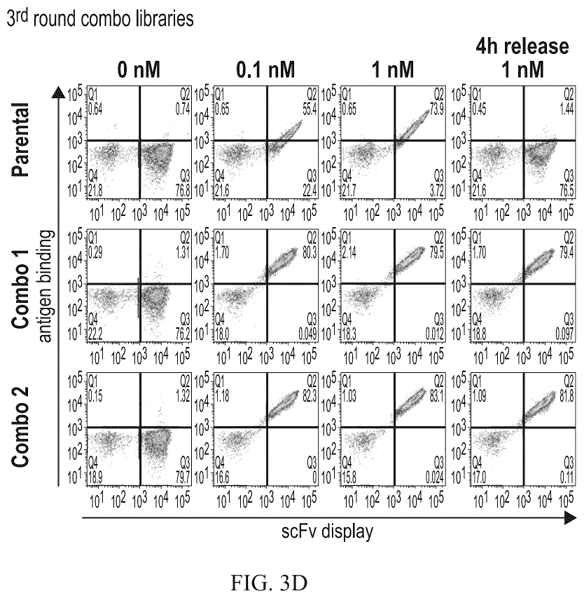

The defined collection of natural CDRs from a known naïve antibody library is purged of sequence liabilities and the reminding liability-free CDRs were inserted these into the lead antibody molecule (parental, not altered, see, FIG. 1A and FIG. 3A) from the original naturally occurring antibody library in one or two sites at a time (LCDR1-2, LCDR3, HCDR1-2) while HCDR3 and framework regions remained constant throughout the process. For example, various LCDR1-2 would be inserted into the lead antibody while the LCDR3/HCDR1-3 remain constant. Hence, a combinatorial antibody library is generated with variable LCDR1-2 and the same LCDR3/HCDR1-3 (see, FIG. 1A, the L1L2 library).

Alternatively, various LCDR3 are used to insert into the lead antibody where the LCDR1/2 and HCDR1-3 remain constant. Hence, a combinatorial antibody library is generated with variable LCDR3 and the same LCDR1-2/HCDR1-3 (see, FIG. 1A, the L3 library).

As another example, various HCDR1-2 may be inserted into the lead antibody while the LCDR1-3/HCDR3 remain constant. Hence, a combinatorial antibody library is generated with variable HCDR1-2 and the same LCDR1-3/HCDR3 (see, FIG. 1A, the H1H2 library).

In one embodiment, various LCDR3 and various HCDR1-2 may be used to insert into the lead antibody where the LCDR1/2 and HCDR3 remain constant. Hence, a combinatorial antibody library is generated with variable LCDR3, variable HCDR1-2 and the same LCDR1-2/HCDR3 (see, FIG. 3A, the Combo 2 library).

In another embodiment, various LCDR1-3 and various HCDR1-2 may be used to insert into the lead antibody where only the HCDR3 remain constant. Hence, a combinatorial antibody library is generated with variable LCDR1-3, variable HCDR1-2 and the same HCDR3 (see, FIG. 3A, the Combo 2 library).

Preferably, all the variable LCDR1-3 and HCDR1-2 and also HCDR3 are liability free.

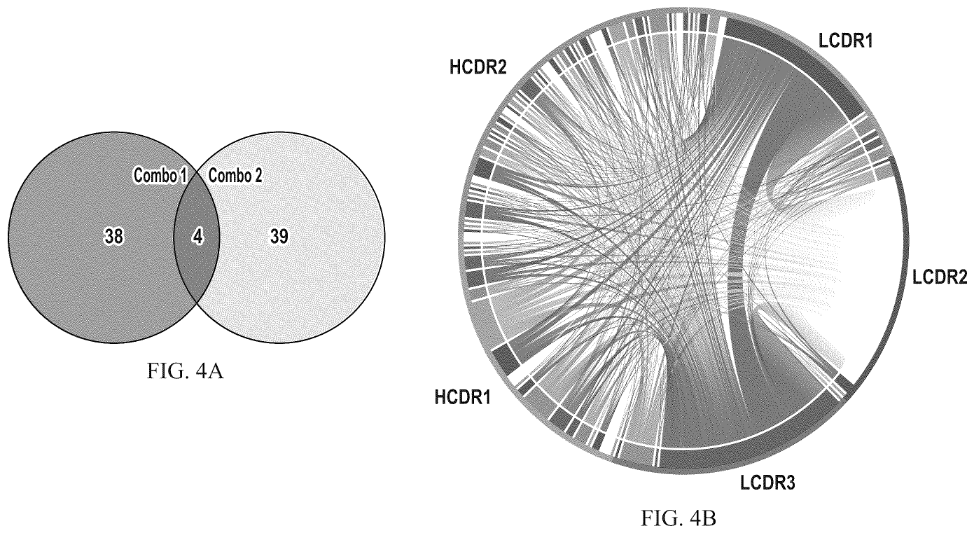

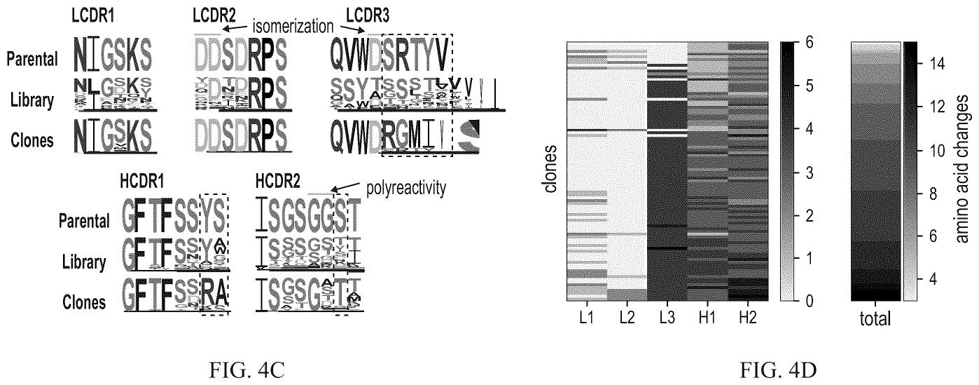

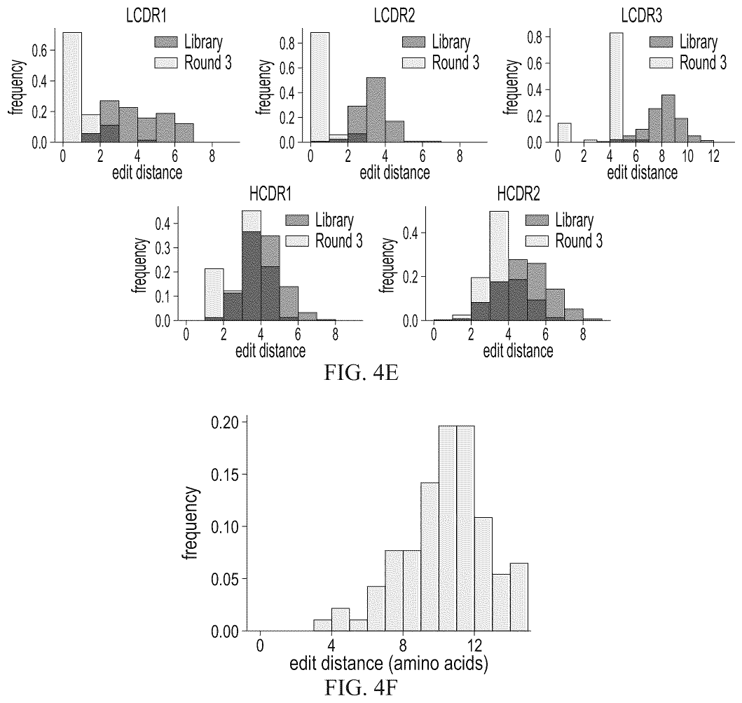

By using the method described, more than 80 unique variants were produced with at least 23 having picomolar affinities and competing with the parental for antigen binding (See, FIG. 4E).

### I. Parental or Lead Antibodies

Disclosed herein are improved methods for affinity maturation of antibodies. The methods involve first selecting a parental or lead antibody with desired binding and/or biological characteristics. After initial identification of lead antibodies that bind to a target of interest, antibody clones may be tested for their ability to achieve a desired biological activity. Such activity may be agonistic or antagonistic. Once a lead's desired activity is identified, the methods disclosed herein enable an antibody to be generated which maintains the epitope specificity and the desired biological activity, while increasing the affinity, which presumably will also increase potency (see, e.g., Rosenfeld et al., 2017; Hurlburt et al. 2020). Any antibody or fragment thereof may be selected for use in the present disclosure. An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single-chain antibody (scFv), single chain Fab (scFab), fusion proteins comprising an antibody portion, humanized antibodies, chimeric antibodies, diabodies, single domain antibodies (also known as nanobodies, e.g., a V_{H} only antibody such as the VhH or VHH antibodies found in camelids), or multispecific antibodies (e.g., bispecific antibodies), or and any other modified configuration of an immunoglobulin molecule comprising an antigen recognition site of the required specificity, including glycosylation variants of antibodies, amino acid sequence variants of antibodies, and covalently modified antibodies. An antibody as disclosed herein includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See, also hgmp.mrc.ac.uk and bioinf.org.uk/abs.

The antibodies described herein may be a full-length antibody, which contains two heavy chains and two light chains, each including a variable domain and a constant domain. Alternatively, the antibodies described herein can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, C_{L} and C₁ domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See, e.g., Bird et al. (1988) Science 242:423-426; and Huston et al. (1988) Proc. Natl. Acad. Sci. USA 85:5879-5883. The same is true of the four domains making up a Fab fragment, which may also be joined together with a synthetic linker to form a single chain Fab (scFab). See Hust et al. BMC Biotechnol. 2007; 7:14.

Any of the antibodies described herein, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

Single-domain antibodies, also known as nanobodies, are also within the scope of the present disclosure. In some embodiments, a single-domain antibody contains only a heavy chain (VHH). Heavy chain only antibodies (HcAb) are naturally produced by camelids and sharks. The antigen binding portion of the HcAb is comprised of the VHH fragment. See, e.g., Vincke et al., Methods Mol Biol. 911:15-26 (2012).

The antibody libraries disclosed herein may contain a population of antibodies of any suitable format. In some embodiments, the antibody library disclosed herein comprise a population of full-length antibodies, which may be of any suitable family (e.g., IgG, or IgA). In other embodiments, the antibody library disclosed herein comprise a population of antigen-binding fragments, for example Fab fragments. In yet other embodiments, the antibody library disclosed herein comprise a population of single-chain antibodies. Alternatively, the antibody library disclosed herein may comprise a population of single-domain antibodies such as VHH fragments.

II. Partially Diversified Antibody Libraries with Liability Free CDRs

In the present disclosure, high affinity antibodies are generated and selected from a partially diversified antibody library. As used herein, the term “partially diversified antibody library” refers to an antibody library comprising a plurality of antibodies, each antibody comprising at least six CDR regions (e.g., V_{H}-CDR1, V_{H}-CDR2, V_{H}-CDR3, V_{L}-CDR1, V_{L}-CDR2, V_{L}-CDR3). Each antibody in said partially diversified antibody library comprises certain “invariant CDR regions” and certain “variant CDR regions.” As used herein, the term “invariant CDR regions” refers to CDR regions, which comprise the same nucleotide and amino acid sequence for every antibody in an antibody library and is derived from the parental or lead antibody. As used herein, the term “variant CDR region” refers to a CDR region whose nucleotide and/or amino acid sequence is unique to an individual antibody or subset of antibodies within the antibody library.

In some embodiments, the partially diversified antibody library may comprise at least two invariant CDR regions (e.g., V_{H}-CDR1, V_{H}-CDR2, V_{H}-CDR3, V_{L}-CDR1, V_{L}-CDR2, or V_{L}-CDR3). In some embodiments, the partially diversified antibody library comprises at least three, four or five invariant CDR regions. Limiting the number of variant CDR regions in a given antibody library decreases the combinatorial diversity between CDR regions and enables more variants at a single CDR region to be generated and screened. Maintaining two or more invariant CDR regions, also increases the chances of finding new variants that bind to the antigen in the same way, which is essential to retaining the biological activity of the parental antibody.

In some embodiments, the parental HCDR3 region is not varied. In some embodiments, V_{L}-CDR3, V_{H}-CDR1, V_{H}-CDR2, and V_{H}-CDR3 are the same for each antibody of the plurality of antibodies of the antibody library. In some embodiments, V_{L}-CDR1, V_{L}-CDR2, V_{H}-CDR1, V_{H}-CDR2, and V_{H}-CDR3 are the same for each antibody of the plurality of antibodies of the antibody library. In some embodiments, V_{L}-CDR1, V_{L}-CDR2, V_{L}-CDR3, and V_{H}-CDR3 are the same for each antibody of the plurality of antibodies of the antibody library. In some embodiments, V_{L}-CDR1, V_{L}-CDR2, and V_{H}-CDR3 are the same for each antibody of the plurality of antibodies of the antibody library. In some embodiments, one of the two or more CDR sequences that are the same for each antibody of the plurality of antibodies of the antibody library is V_{H}-CDR3. In some embodiments, the CDR sequences are derived from a naturally occurring antibody library.

In some embodiments, only true natural CDRs are incorporated into the plurality of antibodies of the antibody library. One advantage of such an approach is that it avoids covariance violations, since the CDRs are known to fold correctly, as they have been derived from natural antibodies. By constructing antibody libraries comprising natural diversity such that the members of the libraries would be prescreened by the immune system for functionality, while excluding members that contain potential liabilities, would be poorly expressed, aggregating and/or poorly folded in a common screening system (e.g., yeast display, phage display, or a folding reporter such as β-lactamase; see, e.g., Saunders et al., Nat. Che Biol., 12:94-101; 1988; and D'Angelo et al., BMC genomics 12, suppl. 1, S1-S5; 2011; or green fluorescent protein; see, e.g., Waldo, et al., Nat. Biotechnol., 17: 691-5; 1999; Cabantous, et al., PLoS ONE., 3:e2387; 2008; and Cabantous, et al., J Struct Funct Genomics, 6:113-9; 2005). The present disclosure thus features, in some embodiments, a method to create extremely diverse, highly functional antibody libraries by combining naturally occurring CDRs, including naturally occurring CDRs containing somatic mutations generated in vivo, within antibody scaffolds such that members of the antibody libraries are expected to be well expressed and/or folded, and lacking liabilities.

As used herein, the term “liability” refers to a motif in an antibody (e.g., located in a heavy chain or light chain CDR region) that would negatively affect one or more desired features of the antibody (e.g., stability, good expression in an expression or display system, proper folding, no or reduced aggregation, solubility, no or reduced integrin binding, no or reduced glycosylation, no or reduced deamidation, no or reduced isomerization, no unpaired cysteine, or no or reduced protease sensitivity, etc.). By virtue of being comprised of highly functional members, such an antibody library would be expected to be functionally much larger than libraries of similar genetic size, in which antibodies are present that contain any of these liabilities. In other words, the antibody libraries disclosed herein would have a much larger effective diversity.

The heavy chain (V_{H}) and/or light chain (V_{L})-CDR1, CDR2, and/or CDR3 sequences for use in generating the antibody libraries described herein and for improving affinity maturation may be further analyzed to remove those that comprise a liability. Exemplary liabilities are listed in Table 1. In some embodiments, naturally occurring CDRs that contain any sequence liabilities are discarded and are not included in the antibody library.

TABLE 1
Liabilities to be Remloved

| Type of Liabilities | Exemplary Motif |
|---|---|
| Glycosylation - impacts stability, solubility, | NXS, → X = Any Amino Acid but Proline |
| half-life, heterogeneity, and effector function. | NXT, → X = Any Amino Acid but Proline |
| | NXC → X = Any Amino Acid but Proline |
| Deamidation - Therapeutic antibodies may | NG, NS, NT, NN, NA, NH, ND, GNF, |
| undergo deamidation during manufacture and | GNY, GNT, or GNG |
| storage leading to protein structural changes, | |
| aggregation, change in pharmacokinetics, | |
| loss of activity and immunogenicity. | |
| Isomerization - Asp residues can undergo | DT, DH, DG, DS, DD |
| isomerization and reported in CDRs. Known | |
| to increase charge heterogeneity | |
| Based on creation of synthetic library, | GG, GGG, RR, VG, VV, VVV, WW, |
| selection against polyspecificity | WWW, YY, WXW (X represents |
| (PSR) and sequencing | any amino acid residue) |
| Single cluster in IL-13 human mAb HCDR3 | FHW |
| which highly aggregating, alanine mutations | |
| increase solubility (aggregation) | |
| Two aromatic tripeptides in HCDR3 mutated | HYF, HWH |
| improve viscosity. Compatible with idea that | |
| 3 consecutive aromatics is bad news and | |
| should be eliminated | |
| Positive charge associated with poor | Net Charge (+1) in |
| developability properties. | LCDR1-3, HCDR1-2 |
| Unpaired cysteine can impact protein folding, | Unpaired Cysteine |
| function and stability. These reactive centers | |
| lead to formation of covalent aggregates and | |
| reduce protein stability | |
| Protease sensitivity | DP, DG, DS, DV, DY, DF, |
| (fragmentation) | DQ, DK, DL, DD |
| Integrin binding site | RGD, RYD, LDV, KGD |
| Lysine glycation site | KE, EK, or ED |
| Metal catalyzed fragmentation | HS, SH, KT, HXS, SXH |
| | (X represents any amino acid residue) |
| Polyspecificity, aggregation | X1X2X3, in which each of X1, |
| | X2, and X3 independent is |
| | F, I, L, V, W, or Y |
| Streptavidin binding motifs | HPQ, EPDW (SEQ ID NO: 117), PWXWL |
| | (SEQ ID NO: 118), GDWVFI (SEQ ID |
| | NO: 119), PWPWLG (SEQ ID NO: 120) |
| | (X represents any amino acid residue) |

In some embodiments, the CDRs (e.g., CDR1, CDR2, CDR3, or a combination thereof) identified as described herein may be experimentally screened or selected for good folding and/or expression and screened or selected against liabilities such as poor folding, poor expression, polyreactivity or aggregation. The selected CDRs may be inserted into complete V domains within the context of the scaffolds. The resultant complete V domains could be further screened and selected for good folding and/or expression, and/or screened and selected against liabilities such as poor folding or expression, polyreactivity or aggregation.

In some instances, V_{H}- and/or V_{L}-CDR sequences comprising one of the liabilities listed in Table 1 (e.g., a glycosylation site, a deamidation site, an isomerization site, an unpaired cysteine, a net charge greater than 1 (e.g., in LCDR1-2 and/or HC CDR1-2), a tripeptide motif containing at least two aromatic residues (which may affect viscosity), a motif that promotes aggregation, (viii) a poly specificity site such as those containing a motif of GG, GGG, RR, VG, W, WV, WW, WWW, YY, or, WXW, in which X represents any amino acid residue; a protease sensitive site (fragmentation sensitive site), or an integrin binding site) can be removed such that the resultant antibody library is free (substantially free or completely free) of members comprising the excluded liability.

Alternatively or in addition, potential glycosylation sites such as lysine glycolation sites may be removed. A glycolation site refers to a site in a protein molecule that can be linked to a sugar molecule via a non-enzymatic process. Exemplary glycolation sites include, but are not limited to, KE, EK, and ED. Additional liabilities include metal catalyzed fragmentation site (e.g., HS, SH, KT, HXS, or SXH, in which X represents any amino acid residue), polyspecificity aggregation site (e.g., having a motif of X1X2X3, in which each of Xi, X2, and X3 is independently F, I, L, V, W, or Y), and streptavidin binding motif (e.g., HPQ, EPDW, PWXWL, in which X represents any amino acid residue, GDWVFI, and PWPWLG).

Substantially free means that the number of a V_{H} and/or V_{H} CDR sequence comprising the liability is less than 20% in the library, e.g., less than 15% or less than 10%.

In some examples, V_{H} and/or V_{L} CDR1, CDR2, and/or CDR3 sequences comprising two or more (e.g., 3, 4, 5, 6, 7, or more) of the liabilities noted above can be removed such that the resultant library is free of (substantially free of or completely free of) members comprising the excluded liabilities. In a preferred embodiment, all of the liabilities listed in Table 1 can be removed such that the resultant library is free of (substantially free of or completely free of) members comprising any of the liabilities.

The resultant heavy chain and/or light chain CDR1, CDR2, and/or CDR3 sequences obtained from naturally-occurring antibodies, either excluding sequences comprising one or more liabilities or maintaining all sequences, can be used as templates to synthesis nucleic acids encoding, and replicating, the CDR sequences for assembling the antibody library and use in the method described herein. Such nucleic acids can be inserted into the corresponding CDR position in the V_{H} and/or V_{L} chain of the lead antibody identified from the naïve naturally occurring antibody library targeting an antigenic epitope.

For example, an initial antibody library may also be sorted for yeast displaying antibodies that have been stained with conformational probes that detect correct antibody folding. Traxlmayr et al, Arch Biochem Biophys. 526(2): 174-80, 2012. Examples of such conformational probes include protein A (Hillson et al, The Journal of experimental medicine. 178(1):331-6, 1993; Akerstrom et al, 1994; J. Imm Methods, 177(1-2): 151-63, 1994; and Roben et al, J. Immunology 154(12):6437-45, 1995) or protein L (Charbit et al, Gene, 70(1): 181-9, 1988; Graille et al, Structure, 9(8):679-87, 2001; and Enever et al, Journal of molecular biology, 347(1): 107-20, 2005), that are able to bind to VH3 and VK domains respectively, and derivatives of indole 3-butyric acid (Alves et al, Langmuir, 28(25):9640-8, 2012; Alves et al, Anal Chem, 84(18):7721-8, 2012; Alves et al, Bioconjug Chem, 25(7): 1198-202, 2014; and Mustafaoglu et al, Biotechnol Bioeng., 112(7): 1327-34, 2015) that binds to the “nucleotide binding site” found in all antibodies (Rajagopalan et al, Proceedings of the National Academy of Sciences of the United States of America, 93(12):6019-24, 1993).

The previous use of conformational probes has been shown to predict high expression and thermostability (Traxlmayr et al, 2012; Shusta et al, J Mol Biol. 292(5):949-56, 1999; Traxlmayr et al, Biochim Biophys Acta., 1824(4): 542-9, 2012; Traxlmayr et al, Protein Eng Des Sel, 26(4):255-65, 2013; and Hasenhindl et al, Protein Eng Des Sel, 26(10):675-82, 2013) in yeast display. This approach selects for antibody fragments that are well expressed and well folded. Rather than positive selection for good display, each individual CDR library can be depleted of CDRs that contain liabilities. For example, adapting screens used for antibody screening (Yang et al, MAbs., 5(5):787-94, 2013; Kelly et al, MAbs, 7(4):770-7, 2015; Kohli et al, MAbs. 7(4):752-8, 2015; Obrezanova et al, MAbs., 7(2)352-63, 2015; Wu et al, Protein Eng Des Sel, 28(10):403-14, 2015; Yang et al, MAbs., 9(4):646-53, 2017; Xu et al, Protein Eng Des Sel, 26(10):663-70, 2013; and Kelly et al, MAbs., 9(7)3036-40, 2017) to yeast display sorting, and isolating those yeast displaying antibodies that correspond to the more “developable” phenotype selects for suitable CDRs that can then be combined to create highly functional libraries.

The sequences encoding functional members of the heavy and/or light CDR1, CDR2, and/or CDR3 can be used as templates for synthesizing nucleic acids coding for such functional members, or used directly. The resultant nucleic acids can then be inserted into the VH and/or VL chain of the lead antibody identified from the naïve naturally occurring antibody library targeting an antigenic epitope as described herein to produce antibody libraries as also described herein. In some embodiments, the antibody library disclosed herein is substantially free of non-functional members, e.g., having less than 10% (e.g., less than 8%, less than 5%, less than 3%, less than 1%, or lower) non-functional members.

### III. Combinatory Antibody Libraries

In some embodiments, the methods for developing a high affinity antibody involves a two-phase diversification strategy that uses defined collections of natural CDRs purged of sequence liabilities. In the first phase, one or more antibody libraries (i.e., Partially Diversified Antibody Libraries) are generated by inserting this defined collection of CDRs into a lead or parental antibody molecule at one or more sites at a time, while at least two of the CDR regions are kept constant in each antibody. The resulting antibodies are then selected for based on their binding affinity for the target and their epitope specificity (i.e., that they maintain the same epitope as the lead or parental antibody). In the second phase, the CDR regions of the selected antibody are combined to create a “combinatorial antibody library”, and the resulting antibodies in said combinatorial antibody are then selected for based on their binding affinities and epitope specificity as well. This two-step affinity maturation strategy allows the diversity at each CDR site to be explored more fully. Library size is often a concern when performing in vitro evolution of any sort since one is limited by the number of transformants that can be conveniently obtained during library generation. Varying only a subset of the CDR regions in each initial partially diversified antibody libraries, allows one to explore the sequence space more effectively, while retaining epitope specificity for the target. As disclosed herein, this methodology results in affinity improvement of an antibody lead to low-picomolar K_{D} values.

In some embodiments, the invention relates to a method of generating a combinatorial antibody library, comprising, generating at least two antibody libraries that each comprises a plurality of antibodies, wherein each antibody of the plurality of antibodies comprises: a V_{H}-CDR1 sequence, a V_{H}-CDR2 sequence, a V_{H}-CDR3 sequence; a V_{L}-CDR1 sequence, a V_{L}-CDR2 sequence, and a V_{L}-CDR3 sequence, wherein each antibody library is generated by selecting two or more CDR sequences as invariant CDR sequences, wherein said invariant CDR sequences are derived from a parental antibody known to bind to the specified target antigen; generating an antibody library comprising a plurality of antibodies, wherein each antibody of the plurality of antibodies comprises: the two or more invariant CDR sequences selected; and a unique combination of variant CDR sequences not selected in; wherein the CDR sequences are derived from the sequences of CDRS found in naturally occurring antibodies; screening each antibody within said antibody library for binding to said specific target antigen; selecting antibodies wherein the selected antibodies bind to the specific target antigen at a higher affinity than said parental antibody; generating a combinatorial antibody library comprising a plurality of antibodies, wherein each antibody of the plurality of antibodies comprises a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence wherein each antibody of the plurality of antibodies comprises a unique combination of variant CDR sequences, wherein the variant CDR sequences are selected from the CDR sequences of the selected antibodies.

In some embodiments, the combinatorial library is generated by combining the CDR regions of selected high affinity antibodies from at least two partially diversified antibody libraries. In some embodiments the combinatorial library is generated by combining the CDR regions of selected high affinity antibodies selected from at least two, three, four, five, six, seven, eight, nine or ten partially diversified antibody libraries. In some embodiments, each of the partially diversified antibody libraries comprises one of the following sets of invariant CDR regions:
(i) (V_{L}-CDR3, VH-CDR1, VH-CDR2, and VH-CDR3);
(ii) (V_{L}-CDR1, VL-CDR2, VH-CDR1, VH-CDR2, and V_{H}-CDR3);
(iii) (V_{L}-CDR1, VL-CDR2, VL-CDR3, and V_{H}-CDR3); and
(iv) (V_{L}-CDR1, VL-CDR2, and V_{H}-CDR3); and
(v) (V_{L}-CDR1, VL-CDR2, VL-CDR3, VH-CDR1 and VH-CDR2)

In some embodiments, each of the partially diversified antibody libraries comprises an invariant V_{H}-CDR3 domain.

The combinatorial antibody library may not contain any invariant CDR regions. In some embodiments, the combinatorial antibody will contain one or more invariant CDR regions. In some embodiments, the combinatorial antibody will comprise one, two, three, four or five invariant CDR regions. In some embodiments, the combinatorial antibody library may comprise an invariant V_{H}-CDR3 domain.

The combinatorial antibody library can be of any form. For example, in some embodiments, the antibody library is a full-length antibody library, a Fab antibody library, a single chain antibody library, or a single domain antibody library. In some embodiments, the antibody library is a human antibody library. It is not necessary that the combinatorial antibody library be of the same type of antibody library as the partially diversified antibody libraries. For example, the combinatorial antibody library might be a full-length antibody library, and the partially diversified antibody libraries used to generate the selected antibodies whose CDR regions make up the combinatorial library may be single chain antibody libraries.

In the present disclosure, the selected one or more CDR sequences that are the same for each antibody of the plurality of antibodies for the methods and antibody libraries (“invariant CDR sequences”) may be (V_{L}-CDR3, V_{H}-CDR1, V_{H}-CDR2, and V_{H}-CDR3) [for the L1L2 library], (V_{L}-CDR1, V_{L}-CDR2, V_{H}-CDR1, V_{H}-CDR2, and V_{H}-CDR3) [for the L3 library], (V_{L}—CDR1, V_{L}-CDR2, V_{L}-CDR3, and V_{H}-CDR3) [for the H1H2 library], (V_{H}-CDR3) [for the Combo 1 library], and (V_{L}-CDR1, V_{L}-CDR2, and V_{H}-CDR3) [for the Combo 2 library]. The CDR sequences are derived from a naturally occurring antibody library wherein the antibodies therein bind a single antigen or a single antigenic epitope and the binding affinity is at least 9.5 nM. The selected one or more CDR sequences form the invariant CDRs in the assembled combinatorial antibody.

In some embodiments, the remaining non-selected CDRs that vary for each antibody of the plurality of antibodies (“variant CDRs”) may be (V_{L}-CDR1 and V_{L}-CDR2) [for the L1L2 library], (V_{L}-CDR3) [for the L3 library], (V_{H}-CDR1 and V_{H}-CDR2) [for the H1H2 library], (V_{L}—CDR1, V_{L}-CDR2, V_{L}-CDR3, V_{H}-CDR1, V_{H}-CRD2) [for the Combo 1 library], and (V_{L}-CDR3, V_{H}-CDR1, V_{H}-CDR2) [for the Combo 2 library].

In some embodiments, the CDR sequences are free of one or more of the following sequence liabilities: (i) a glycosylation site, (ii) a deamidation site, (iii) an isomerization site, (iv) unpaired cysteine, (v) net charge greater than 1, (vi) a tripeptide motif containing at least two aromatic residues, (vii) a motif that promotes aggregation, (viii) a poly specificity site; (ix) a protease sensitive site, (x) an integrin binding site, (xi) a lysine glycosylation site, (xii) a metal catalyzed fragmentation site, (xiii) a poly specificity aggregation site; and (xiv) a streptavidin binding motif.

In some embodiments, the glycosylation site may comprise the motif NXS, NXT, or NXC, in which X represents any naturally-occurring amino acid residue except for proline; the deamidation site may comprise the motif of NG, NS, NT, NN, NA, NH, ND, GNF, GNY, GNT, or GNG; the isomerization site may comprise the motif of DT, DH, DS, DG, or DD; tripeptide may be HYF or HWH; the motif that promotes aggregation may comprise the motif of FHW; the poly specificity site may comprise the motif GG, GGG, RR, VG, W, WV, WW, WWW, YY, or WXW, in which X represents any amino acid residue; the protease cleavage site may comprise the motif of DX, in which X is P, G, S, V, Y, F, Q, K, L, or D; the integrin binding site may comprise RGD, RYD, LDV, or KGD; the lysine glycosylation site may comprise KE, EK, or ED; the metal catalyzed fragmentation site may comprise the motif of HS, SH, KT, HXS, or SXH, in which X represents any amino acid residue; the poly specificity aggregation site may comprise a motif of X1X2X3, wherein each of Xi, X2, and X3 independently is selected from the group consisting of F, I, L, V, W and Y; and the streptavidin binding motif may comprise the motif HPQ, EPDW, PWXWL, in which X represents any amino acid residue, GDWVFI, or PWPWLG.

### IV. Methods of Selecting High Affinity Antibodies

Described herein, in various embodiments, are methods of affinity maturation of a parental antibody, such that one or more high affinity antibodies to a specified epitope are selected. Such methods may include the generation and screening of a partially diversified antibody library. Such methods may also include the generation and screening of a combinatorial antibody library.

The antibody libraries disclosed herein can be made using known techniques in the art. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

Any of the antibody libraries described herein may be used to screen for antibodies having binding specificity to an antigen of interest. Antibodies encoded by the nucleic acids in the library can be expressed and displayed using a suitable expressing/display system, for example, a cell-free display system (e.g., ribosome display), a phage display system, a prokaryotic cell-based display system (e.g., bacterial display), or a eukaryotic cell-based display system (e.g., yeast display or mammalian cell display). In certain embodiments, the antibody libraries are expressed and displayed on yeast cells. In other embodiments, the antibody libraries are expressed and displayed on phage particles (phage display). In other embodiments two or more display systems are used, e.g., phage display followed by yeast display.

The library of antibodies may be expressed/displayed in a suitable system, e.g., those described herein, in any format. Examples include intact antibodies (full-length antibodies), antigen-binding fragments thereof (e.g., Fab), or single chain antibodies (scFv or scFab). Phage display is a protein display format using bacteriophages (e.g., phage fl, fd, and M13). In this system, at least one antibody chain (e.g., the heavy chain and/or the light chain) is typically covalently linked to a bacteriophage coat protein, for example, a gene III protein, a gene VIII protein, or a major coat protein (see, e.g., WO 00/71694). Phage display is described, for example, in U.S. Pat. No. 5,223,409; Smith (1985) Science 228: 1315-1317; WO 92/18619; WO 91/17271; WO 92/20791; WO 92/15679; WO 93/01288; WO 92/01047; WO 92/09690; WO 90/02809; de Haard et al. (1999) J Biol. Chem 274: 18218-30; Hoogenboom et al. (1998) Immunotechnology 4: 1-20; Hoogenboom et al. (2000) Immunol Today 2:371-8; Fuchs et al. (1991) Bio/Technology 9: 1370-1372; Hay et al. (1992) Hum Antibod Hybridomas 3:81-85; Huse et al. (1989) Science 246: 1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9: 1373-1377; and Hoogenboom et al. (1991) Nuc Acid Res 19:4133-4137.

Bacteriophage displaying the protein component can be grown and harvested using standard phage preparatory methods, e.g., PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced.

In other embodiments, a eukaryotic expression/display system, e.g., yeast cells or mammalian cells, can be used for expressing and displaying the library of antibodies as described herein. Yeast display is a protein display format, in which a protein component (e.g., an antibody component) is linked to a yeast cell wall protein (e.g., Agalp or Aga2p) directly or indirectly. In some instances, one chain of an antibody can be covalently fused to the yeast cell wall protein for direct display. In other instances, the association between an antibody component and a yeast cell wall component can be mediated by an intermediate agent. Yeast display is described in, e.g., Cho et al., J. Immunol. Methods, 220(1-2): 179-188, 1998; Boder et al, Methods Enzymol. 192(2):243-248, 2000; van den Beucken et al., FEBS Lett 546(2-3):288-294, 2003; and Boder et al, Arch Biochem Biophys 526(2): 99-106, 2012. To screen an antibody library as described herein for isolating antibodies capable of binding to a target antigen, the library of antibodies can be in contact with the target antigen under suitable conditions allowing for antibody-antigen binding. Phage particles or host cells displaying antibodies binding to the target antigen can be isolated, for example, by retention or a support member on which the target antigen is immobilized, amplified if needed, and the nucleic acids coding for the displayed antibodies can be determined. The screening process can be repeated multiple time, and display systems can be used in combination. When needed different antigens can be used for selecting antibody members having desired binding specificity or for negative selection to exclude antibody members having binding activity to a non-target antigen.

The screening of the antibodies derived from the libraries described herein can be carried out by any appropriate means. For example, binding activity can be evaluated by standard immunoassay and/or affinity chromatography. Determining the ability of candidate antibodies to bind therapeutic targets can be assayed in vitro using, e.g., a BIACORE™ instrument, which measures binding rates of an antibody to a given target antigen based on surface plasmon resonance. In vivo assays can be conducted using any of a number of animal models and then subsequently tested, as appropriate, in humans. Cell-based biological assays are also contemplated.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.). In order that the invention described may be more fully understood, the following examples are set forth. The examples described in this application are offered to illustrate the methods and compositions provided herein and are not to be construed in any way as limiting their scope.

### EXAMPLES

Affinity maturation is a necessary step for the development of potent therapeutic molecules. Technology for introducing diversity into proteins has long existed and has been extensively used to evolve molecules with desired characteristics, including affinity maturation of antibodies in vitro (Kang, Jones et al. 1991, Hawkins, Russell et al. 1992, Marks, Griffiths et al. 1992, Jackson, Sathe et al. 1995, Yang, Green et al. 1995, Low, Holliger et al. 1996, Schier and Marks 1996, Schier, McCall et al. 1996, Thompson, Pope et al. 1996, Hanes, Jermutus et al. 1998, Hemminki, Niemi et al. 1998, Wu, Beuerlein et al. 1998, Boder, Midelfort et al. 2000, Hanes, Schaffitzel et al. 2000, Rader, Popkov et al. 2002, Lu, Shen et al. 2003, Rajpal, Beyaz et al. 2005, Garcia-Rodriguez, Levy et al. 2007, Lippow, Wittrup et al. 2007, Yang, Yoon et al. 2007, Barderas, Desmet et al. 2008, Gonzalez-Munoz, Bokma et al. 2012, Votsmeier, Plittersdorf et al. 2012, Thakkar, Nanaware-Kharade et al. 2014, Frigotto, Smith et al. 2015, Hu, Hu et al. 2015, Tiller, Chowdhury et al. 2017, Cannon, Shan et al. 2019).

The approach of mimicking natural CDR diversification to improve affinity shares similarities with previously published works (Julian, Li et al. 2017, Tiller, Chowdhury et al. 2017) with the difference that instead of using degenerations to simulate the frequency of naturally occurring amino acids at a given position, complete natural CDR sequences from the human repertoire were rescued and synthesized, taking advantage of recent advances in synthetic biology that enabled large-scale and low-cost production of oligonucleotide pools (Kosuri and Church 2014). In this way, only true natural CDRs are incorporated, rather than diversity that merely resembles natural CDR diversity. One possible advantage of such an approach is that it avoids covariance violations (Chou, Némethy et al. 1989, Du, Wei et al. 2003), since the CDRs are known to fold correctly, as they have been derived from natural antibodies. Of note, the sequenced libraries used for our source diversity were derived from naïve CD19+ B cells, with most V regions having fewer mutations when compared to the fully mature IgG repertoire.

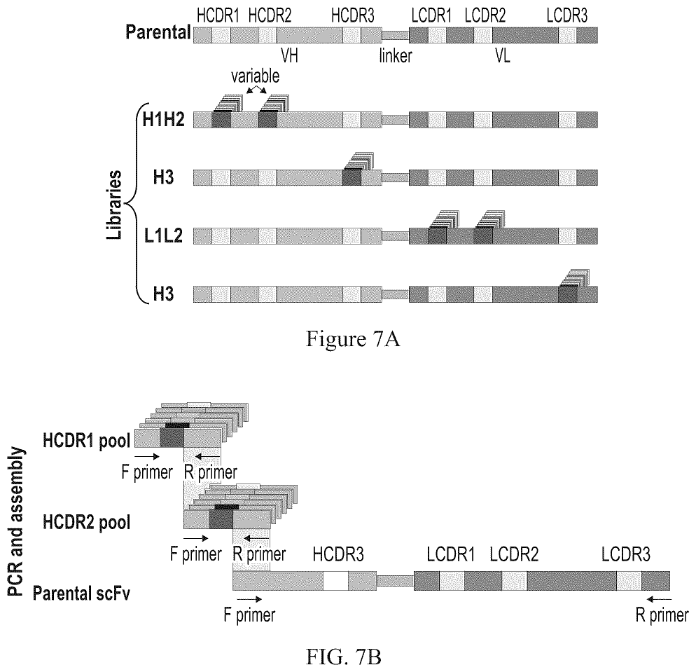

The present disclosure demonstrates the affinity improvement of lead antibodies to low-picomolar K_{D} values, while retaining identical epitope binding, and with the reduction of the number of sequence liabilities. This is based on the use of a new diversification strategy that uses defined collections of natural CDRs purged of sequence liabilities. First, CDR sequences were identified by NGS of a naïve library (Erasmus, D'Angelo et al. 2021, see, U.S. Pat. No. 10,954,508, the content of which is hereby incorporated by reference in its entirety). The identified individual CDRs were chemically synthesized without degenerations, and with all sequence liabilities removed, and inserted into the lead molecule in one or two sites at a time (Phase I: LCDR1-2, LCDR3, HCDR1-2) (see, FIG. 1A and FIG. 7A). HCDR3 and framework regions were kept constant throughout the process. Alternatively, HCDR3 was mutationally scanned, in this approach each amino acid position in the HCDR3 sequence was mutated to 19 amino acids (excluding cysteine) for a total of Y×19 oligonucleotides, where Y is the number of HCDR3 amino acids to be mutated (see, FIG. 7A). After selecting improved affinity variants using yeast display, the resulting variable regions of each library were combined during Phase 2, producing one or two Combo libraries, and these combo libraries are further selected (see, FIG. 1A and FIG. 7A).

### Example 1: Parental Antibody Identification and CDR Design for Three Phase 1 Libraries

An antibody against a monomeric human protein of therapeutic interest was chosen to be affinity matured using the improved affinity maturation method. The antibody, comprising a VH3 heavy chain and a Vλ3 light chain, was initially identified by biopanning a naïve human scFv phage display library (Erasmus, D'Angelo et al. 2021). Analysis of the CDRs revealed three sequence liabilities with the potential of affecting downstream clinical development: two distinct aspartate isomerization sites, which can lead to chemical degradation and loss of potency (Sydow, Lipsmeier et al. 2014), at LCDR2 and LCDR3, and a GG non-specificity motif (Kelly, Le et al. 2018) at HCDR2. The scFv affinity (K_{D}) was determined to be 9.5 nM (kₐ=4.6×10⁵ M⁻¹s⁻¹; k_{d}=4.3×10⁻³ s⁻¹) by surface plasmon resonance (FIG. 1B).

Deep sequencing data (MiSeq and NovaSeq) from the same antibody library were used to identify CDRs that could be shuffled into the parental sequence for affinity maturation. For LCDR1-2 only, CDR sequences coming from the Vλ3 germline family were considered and for HCDR1-2 only, sequences coming from the VH3 family were considered, assuming that these sequences, since they belonged to the same germline genes as the parental antibody, would be better tolerated and minimize any potential structural disruption. For LCDR3, however, all k light chain LCDR3 sequences were included, regardless of the specific family or germline since, in theory, the CDR3 region should be able to support a more diverse set of sequences.

To minimize the chance of selecting antibodies with unfavorable developability profiles, sequences containing undesirable liabilities such as glycosylation sites and unpaired cysteines were eliminated (see, Table 2 for the full list of sequence liabilities removed from CDRs designed for the affinity maturation libraries). Every CDR identified containing any of the listed motifs were discarded. Finally, the identified CDRs were generated with flanking framework sequences matching the parental antibody and produced using array-based DNA synthesis (Agilent). This allowed the rescue of the full diversity at each individual CDR site by using framework primers.

TABLE 2
List of Undesirable Sequence Liabilities

| Liability | Motifs | Reference |
|---|---|---|
| N-linked | NxS and NxT, where x | Gavel and von |
| glycosylation | is not a Pro | Heijne 1990; Kumar |
| | | and Singh 2016 |
| Asn deamidation | NG, NS, NT, NN | Sydow, Lipsmeier |
| | | et al. 2014; Kumar |
| | | and Singh 2016 |
| Asp isomerization | DG, DS, DD | Sydow, Lipsmeier |
| | | et al. 2014 |
| Polyreactivity | GG, RR, VG, VV, YY, | Kelly, Le et |
| | WW, WxW | al. 2018 |
| Aggregation | FHW | Wu, Luo et al. |
| | | 2010; Bethea, |
| | | Wu et al. 2012 |
| Unpaired Cys | Odd Cys number | Kumar and |
| | | Singh 2016 |
| Proteolysis | DP | Vlasak and |
| | | Ionescu 2011 |

### Example 2: Construction of Three Phase 1 Libraries

Library size is often a concern when performing in vitro evolution of any sort since one is limited by the number of transformants that can be conveniently obtained during library generation: 10⁹-10¹⁰ for phage and E. coli display and 10⁸-10⁹ for yeast display in S. cerevisiae. The present disclosure opted for a step-wise approach (Hemminki, Niemi et al. 1998) since it would allow exploring the sequence space more effectively: in phase 1, LCDR1 was combined with LCDR2, HCDR1 and HCDR2, and LCDR3 was left by itself (Table 3), but there was no reason to believe that this was somehow the optimal approach. It was done so out of what seemed to be the most convenient combinations for library building. These libraries allowed generation of more transformants than the maximum combinatorial diversities for each (theoretical diversity ranging from 1.7×10⁵ to 3.2×10⁷). Also, by fixing at least four parental CDRs, including the all-important HCDR3, in each library, search space was decreased, increasing the chances of finding new variants binding to the antigen in the same way, essential to retaining biological activity. In addition to the incorporated CDR diversity, each library also included the parental CDR sequences at the same abundance as the other introduced CDRs, even if they contained sequence liabilities. This was to ensure retained activity in the case that particular parental CDR sequences were essential for binding.

TABLE 3
Number of Different Sequences introduced
in each CDR position of LC and HC

| Family | Region | CDRs | Library | Diversity | Transformants |
|---|---|---|---|---|---|
| Vλ3 | LCDR1 | 1,155 | L1L2 | 498,960 | 1.26 × 10⁷ |
| | LCDR2 | 432 | | | |
| Vλ | LCDR3 | 166,196 | L3 | 166,196 | 1.23 × 10⁷ |
| VH3 | HCDR1 | 9,874 | H1H2 | 31,685,666 | 7.4 × 10⁷ |
| | HCDR2 | 3,209 | | | |

The five different collections of CDR sequences (LCDR1, LCDR2, LCDR3, HCDR1, and HCDR2) were amplified with specific primers by PCR using Q5 polymerase (NEB #M0491L). The remaining regions were amplified from the parental scFv and assembled with the CDRs by PCR (see, FIGS. 1A and 1C). Libraries were assembled by combining the newly produced CDR pools with the remaining parts of the scFv. For example, the L1L2 library was assembled by (1) amplifying the LCDR1 and LCDR2 with the flanking frameworks from the synthetic oligo pool, (2) amplifying the remaining parts of the scFv from the parental clones, (3) assembling the produced fragments by overlap PCR, and (4) transforming the produced scFv cassettes into S. cerevisiae along with the digested yeast display vector (FIG. 1C). The scFv amplicons from each library were transformed into yeast along with the yeast display vector pSYD previously digested with the enzymes BssHII and NheI (NEB #R0199S and #R0131S) by electroporation using method described previously (Benatuil, Perez et al. 2010).

The present disclosure chose to use scFv yeast display because it provides high precision in retrieving the desired population when combined with flow cytometry. However, it was expected this diversification approach was equally effective in the phage or ribosome display context and one could even expand possibilities due to larger library sizes. However, experimental design during selection would have to be adapted to these platforms since they are expected to show different behavior due to their monovalent nature as opposed to the multivalent nature of yeast: whereas a polyvalent yeast cell exists in a continuum from antigen saturation to no antigen binding that varies over time in proportion to the antibody off-rate, monovalent systems can only exist in the binary bound or not-bound states. This difference can be overcome, for example, by using a larger number of displaying particles and relying on the population binding decay over time as opposed to the single cell decay. The present disclosure routinely use the scFv format for antibody discovery and engineering and have found conversion to the IgG format occurs with 70-90% success. However, if conversion is a concern, the techniques described here would be easily applied using a Fab display system.

### Example 3: Antibody Selection by Yeast Display Using Three Phase 1 Libraries

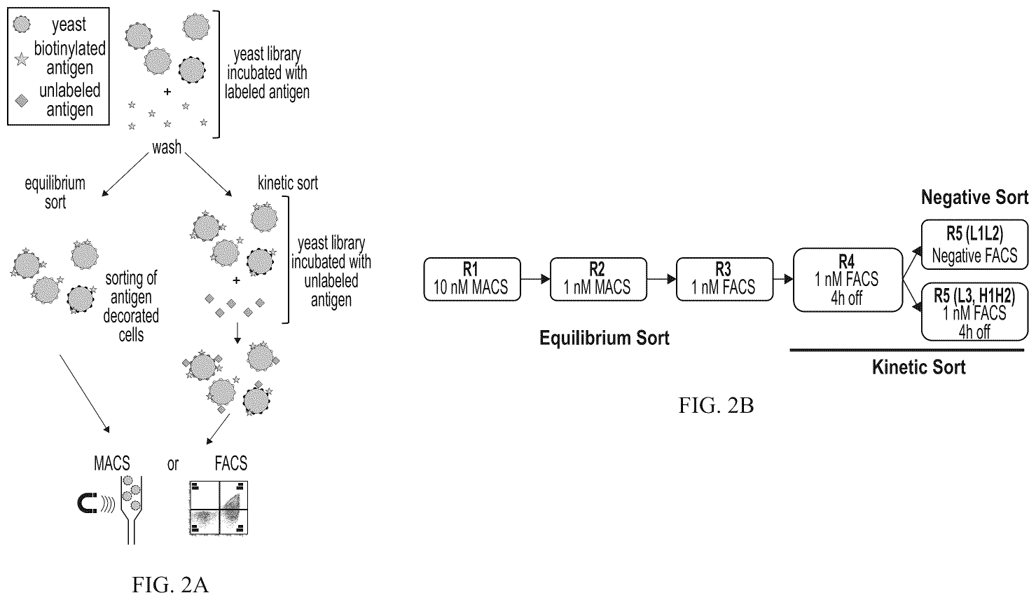

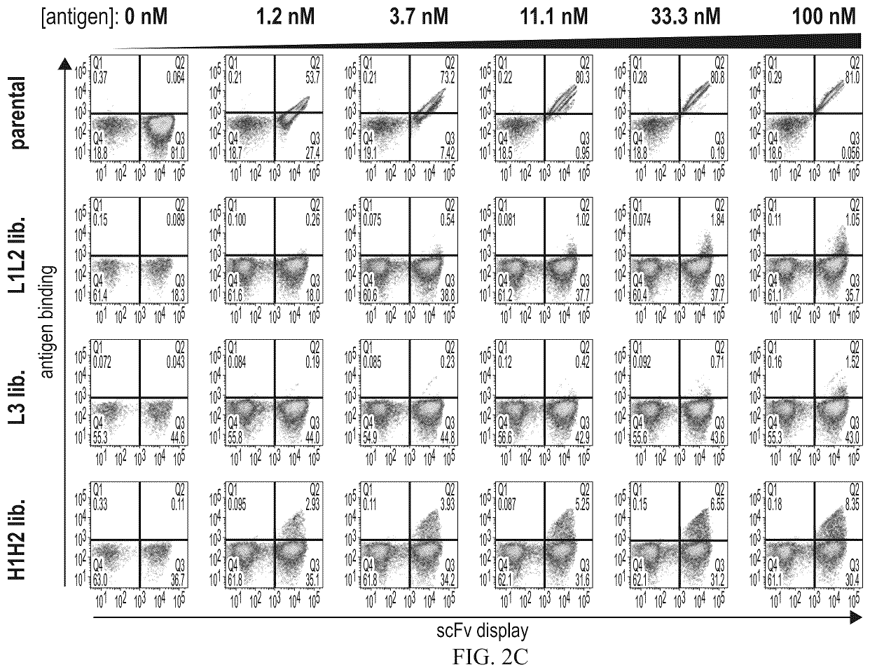

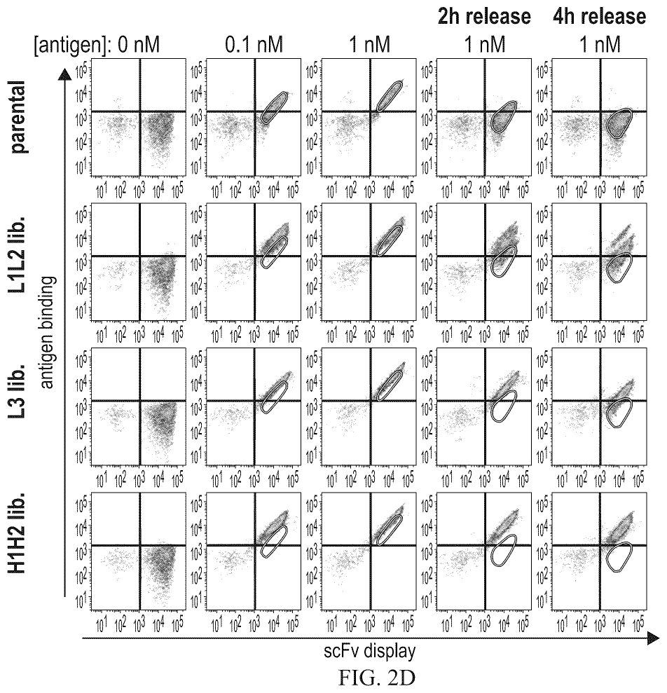

Yeast display selections were performed as in Ferrara et al (2012). Briefly, cells were induced in selective media containing 2% galactose overnight at 20° C. 10⁵ induced cells are washed twice with cold washing buffer (PBS pH 7.4 0.5% BSA) and incubated at room temperature with the biotinylated antigen diluted in PBS. Two different selection strategies were used in this phase: equilibrium selection and kinetic selection (FIGS. 2A-2B) (Boder and Wittrup 1998, Boder, Midelfort et al. 2000). Equilibrium selection, the more traditional approach, was performed by incubating the scFv-displaying yeast cells with a defined concentration of labeled antigen (biotinylated, in this case) and sorting labeled cells immediately after reaching equilibrium. Incubations are often performed with decreasing antigen concentrations as the selections round progress. After the biotinylated antigen incubation step the cells are washed and stained promptly with the anti-SV5 labeled with PE (phycoerythrin; labels cells displaying scFv) and streptavidin labeled with Alexa Fluor 633 (Thermo Scientific; labels cells bound to biotinylated antigen) and then cells binding the antigen are sorted either by FACS (fluorescence-activated cell sorting) or MACS (magnetic-activated cell sorting). For the MACS procedure, paramagnetic beads coated with streptavidin were used (Miltenyi Biotec).

Decreasing the antigen concentration cannot be carried out indefinitely since the displayed antibodies on the yeast surface will deplete antigen from the solution before reaching equilibrium (VanAntwerp and Wittrup 2000). Avoiding this requires the use of large and impractical incubation volumes with small numbers of cells, to ensure minimal antigen is removed and the effective concentration remains constant. An alternative to equilibrium selection is kinetic selection: scFv-displaying yeast cells are incubated with the labeled antigen, washed, incubated with unlabeled antigen (10× more concentrated than the biotinylated antigen) to select only clones with stable binding to the antigen (slow off-rate—k_{d}). The unlabeled antigen is used to prevent rebinding of the displaced labeled antigen. After a defined period, cells still bound to the labeled antigen were stained and sorted as described before.

The present disclosure performed an initial flow cytometric assessment of the libraries using decreasing antigen concentrations (FIG. 2C). The light chain libraries, L1L2 and L3, showed a small population binding to the antigen even at the highest concentration used: at 100 nM binding populations of 3% and 1.5% respectively. As for the heavy chain library, H1H2, significant binding could be observed from 1.2 nM (2.9% of the population) to 100 nM (8.3% of the population), suggesting higher improvement potential for the heavy chain CDRs as opposed to the light chains for this particular clone.

Given the size of each of these libraries, the present disclosure performed first and second rounds of selection using magnetic-assisted cell sorting (MACS) at antigen concentrations of 10 nM and 1 nM, respectively. This allowed the present disclosure to label and sort a larger number of cells than what would be practical using a flow cytometer. For subsequent rounds, the present disclosure used fluorescence activated cell sorting (FACS) to enable more precise sorting of the cells of interest. After the first three rounds of equilibrium sorting, the present disclosure performed two rounds of kinetic sorting with 4 hours competition for the L3 and H1H2 libraries. Only one 4 h kinetic sort was performed for the L1L2 library, and the final round was performed as a negative sort, where the population was incubated only with the secondary reagents and negative cells were sorted—this was done out of concern that polyreactive antibodies may be enriched from this library, since weak positivity was identified in the absence of antigen in the kinetic sort of this library (but not the others) (FIG. 2D).

An assessment of the population obtained for each of these libraries after five rounds show that in all cases a significant improvement of affinity can be observed (FIG. 2D): all generated populations show significant binding to the antigen even 4 hours after the labeled antigen has been removed from solution, a time at which the parental antibody shows minimal binding. Even though affinities of individual clones have not been assessed at this stage, it is not unreasonable to assume that, given the yeast display staining profile, one could already find binders with satisfactory affinities depending on the requirements of the project.

### Example 4: Phase 2 Combo Libraries Construction from Three Phase 1 Libraries

After selecting CDRs at each position in the three initial libraries that showed improved binding, the present disclosure combined the selection outputs with the goal to further improve binding towards still slower off-rates. Two “combo” libraries were assembled by PCR: the first (Combo 1—2.27×10⁸ transformants) was created by combining the output of all three libraries (L1L2, L3, and H1H2), while the second (Combo 2—1.04×10⁸ transformants) omitted the L1L2 output and used the parental CDRs at LCDR1 and LCDR2 (FIG. 3A). Again, an approach combining equilibrium and kinetic sorts was used (FIG. 3B) with two successive rounds of kinetic sorting at 4 h and 16 h with unlabeled antigen respectively, followed by a final equilibrium round at 0.1 nM antigen concentration.

An initial assessment showed that straight after transformation the libraries were already showing binding to the antigen even at 0.1 nM—51.8% and 62.9% of the population for Combo 1 and 2, respectively (FIG. 3C). After three rounds of sorting, the difference between the combo libraries and the parental antibody is striking (FIG. 3D): at 0.1 nM>80% of the yeast population bound to the antigen for both libraries as opposed to 55% for the parental antibody; when stained with 1 nM of antigen and destained for 4 h, both combo libraries show very little signal loss compared to the staining at 1 nM of antigen with no destaining period, whereas the parental completely lost binding under these conditions.

### Example 5: Sequence Screening of Affinity Matured Clones

To evaluate the clones obtained the present disclosure converted the populations obtained after the 3rd round of selection of the Combo libraries to an scFv-Fc format to facilitate affinity screening. The scFv from the final population (round 3, combo libraries) was bulk cloned into a yeast expression vector containing a human IgG1 Fe region to be expressed in the scFv-Fc format. For this, the scFv region was amplified by PCR, digested with BssHII and NheI restriction enzyme (New England Biolabs) and cloned into the pDNL9 vector. 92 clones (45 from Combo 1 and 47 from Combo 2) were sequenced and 81 unique sequences were identified: 38 from Combo 1, 39 from Combo 2, and 4 in both libraries (FIG. 4A). These unique sequences were often formed by different combinations of the same CDRs (FIG. 4B): 12, 5, 5, 20, and 45 unique CDRs were found respectively for LCDR1-3 and HCDR1-2 (Table 4). Out of the 92 sequenced clones, 81 unique ones were identified. Interestingly, the heavy chain CDRs were more diverse than the light CDRs—this relates to the observed binding pattern of the first naïve libraries that showed higher binding signal for the H1H2 library (FIG. 2C) suggesting that the heavy CDRs indeed were more tolerant to sequence changes.

TABLE 4
Number of Unique CDRs Identified

| Region | Unique |
|---|---|
| LCDR1 | 12 |
| LCDR2 | 5 |
| LCDR3 | 5 |
| HCDR1 | 20 |
| HCDR2 | 45 |

Sequence comparison of the CDRs present in the parental antibody, the maturation libraries, and the identified clones (FIG. 4C) shows that in many cases the amino acid in the parental antibody was optimal since after several rounds of selections the clones converge back to the same sequence. While there was high tolerance for diversity overall, LCDR1 and LCDR2 seemed to tolerate far fewer changes than the other CDRs and for LCDR2, almost all clones converge back to the parental sequence, justifying the inclusion of the parental CDR sequences in the libraries. In LCDR3, despite introducing a diversity of 166,196 different CDRs coming from all k germlines, only identified 5 different sequences were identified with the most abundant being present in 76 out of the 92 clones sequenced; there was low tolerance for length diversity. As for HCDR1 and HCDR2, much of the diversity found is concentrated in a few positions/hotspots, suggesting that these may be less relevant for binding (e.g.: position 6 at both HCDR1 at HCDR2), while some other positions showed clear convergence to an amino acid different to the parental (FIG. 4C, dashed squares). Additionally, two of three isomerization motifs may be eliminated, and the GG polyreactivity motif may be eliminated, resulting in the potential elimination of three of four sequence liabilities.

The number of CDR mutations in the antibodies ranged from only 3 total mutations up to 15 amino acid changes (FIG. 4D). The most frequent number of mutations was 11-12 (FIG. 4E). A caveat here being that about half the clones came from the Combo 2 library in which LCDR1 and LCDR2 were kept constant. That said, only one LCDR1 had 4 mutations from parental with all other having 2 or less (FIG. 4E). For LCDR3, the dominant sequence had 4 mutations from parental, and for HCDR1 and HCDR2, 3 mutation was the most frequently observed change.

None of the affinity matured antibodies had sequence liabilities in LCDR1, HCDR1, HCDR2, and HCDR3 (this last one remained unchanged from the parental). The GG that was present in parental HCDR2 was most often replaced by GS, GA, GT, and GD (present in 21, 19, 13, and 11 unique clones respectively). For LCDR3, 67 out of the 81 clones did not have any liabilities, 13 had the same sequence as the parental and therefore had a DS aspartate isomerization site, and one had the same isomerization site plus a VV polyreactivity motif—probably arising from a synthesis or PCR error since this CDR was not included in the initial design. For LCDR2, only 1 of the 81 unique clones did not have the DD aspartate isomerization motif, replaced by an ED, while 72 had the exact sequence of the parental LCDR2 harboring the liability. 8 others were single mutations from the parental sequence and still harbored the same motif (again, likely coming from synthesis/PCR errors). In summary, 68 affinity matured clones had a single CDR liability (Asp isomerization), 12 clones had 2, and 1 clone had 3, showing that the method can be effectively used to reduce developability issues simultaneously with affinity maturation.

In the present disclosure, the affinity of their lead was improved while sequence liabilities that were present in LCDR3 and HCDR2 were abolished. Most LCDR2 still had the original isomerization motif, but one liability-free sequence was identified for this site and could be used in a final molecule if this liability proved problematic. In theory, one could omit original parental CDRs containing sequence liabilities in the affinity maturation libraries, but at the risk of being unable to mature the clone since the eliminated motif may be essential for binding, as seems to be the case for the DD in the LCDR2 of this clone. Removing liabilities from a therapeutic mAb has a series of benefits: de-risking the lead upfront can improve developability and minimize chances of clinical failure due to poor molecule properties (Jain, Sun et al. 2017). In addition, increased immunogenicity risk (e.g., glycosylation) and loss of potency (e.g., asparagine deamidation) are Critical Quality Attributes (CQA's) that require tracking and risk management strategies to be put in place, increasing the resource burden on the development process and on the overall drug life cycle. Carrying out separate engineering campaigns to independently improve affinity and developability is not only time and resource consuming but can also lead to an endless developmental loop since affinity improvements can lead to developability problems and vice-versa (Pepinsky, Silvian et al. 2010, Wu, Luo et al. 2010, Tiller, Li et al. 2017). By using defined collections of natural CDR sequences lacking sequence liabilities the motifs present in the final antibody can be easily modulated.

It is noteworthy that in the affinity maturation of this antibody, the present disclosure observed that HCDR1 and HCDR2 were much more amenable to substitutions than LCDR1 and LCDR2. This was reflected in the naïve L1L2 libraries showing significantly less binding than the H1H2 in the beginning of the process, and also in the final number of different CDRs identified for each position at the end. Hypothesizing that the lack of significant improvement with the L1L2 library may reflect the presence of (close to) optimal sequences for these CDRs, the present disclosure built a second combo library where parental sequences were used for LCDR1-2. The performances of both combo libraries was equivalent during sorting, and affinity measurements also showed similar results (Example 8). In fact, 17 out of the 45 unique clones identified from the first combo library had the unmutated parental LCDR1-2 as well, confirming the importance of these CDRs in binding—although the possibility of cross-contamination between the first 3 libraries (L1L2, L3, H1H2) cannot be definitively excluded.

### Example 6: Epitope Screening of Affinity Matured Clones

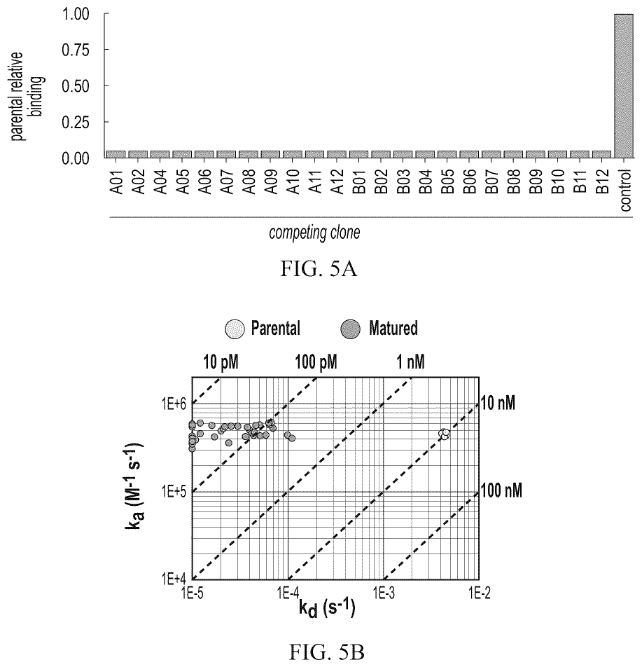

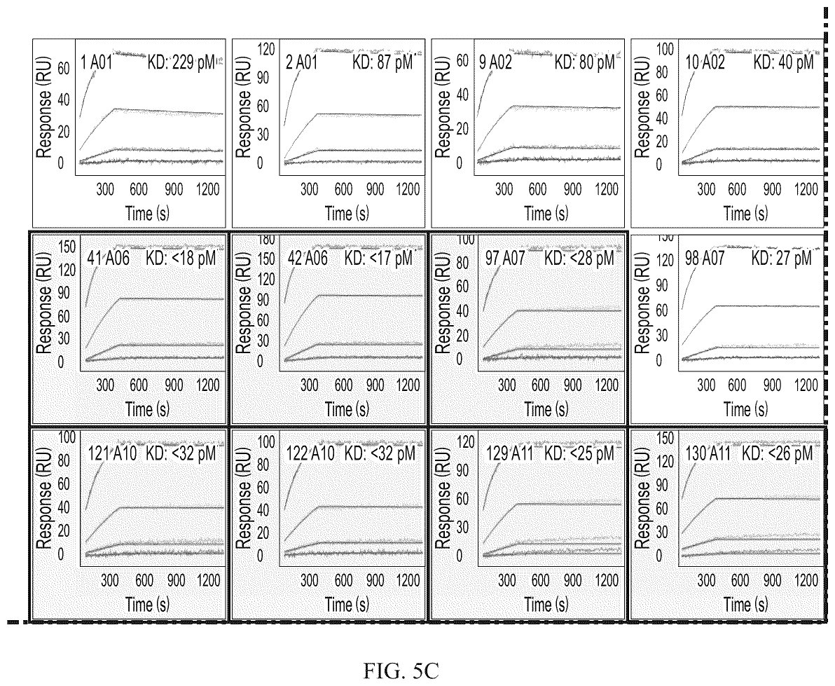

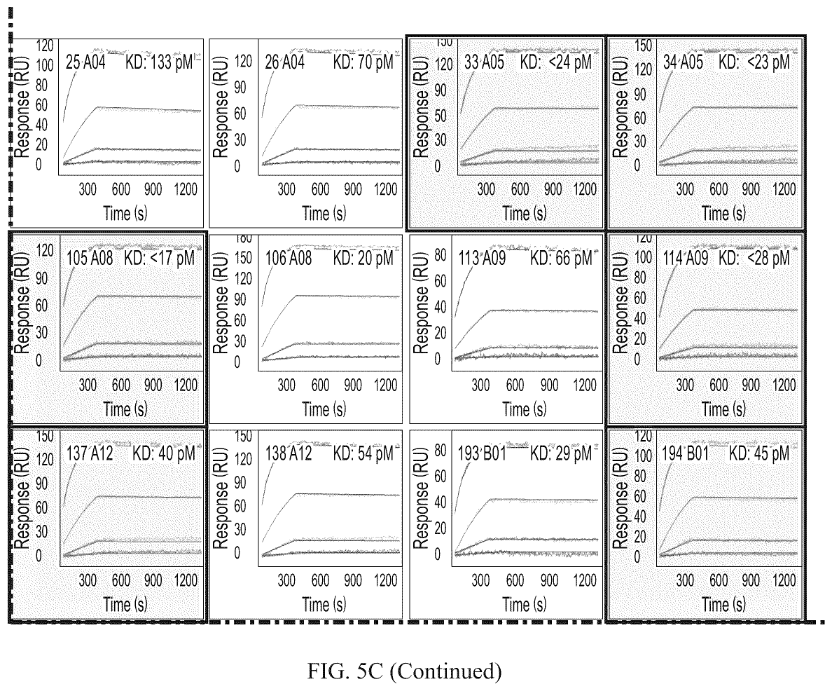

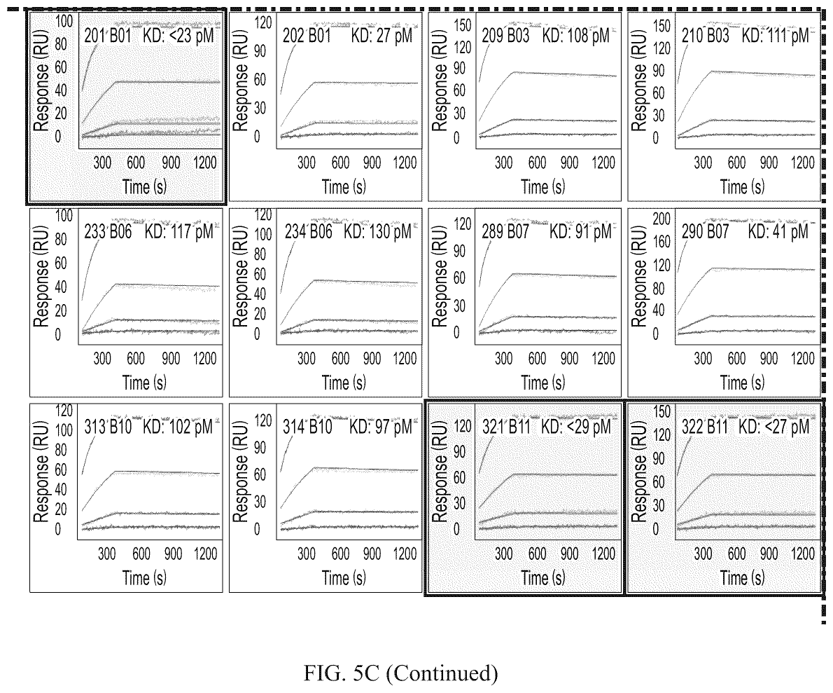

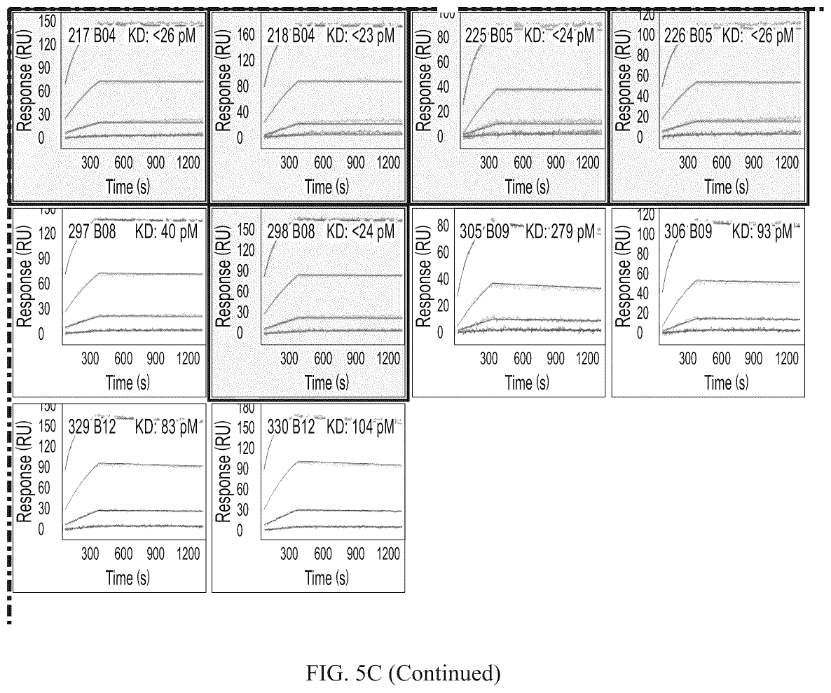

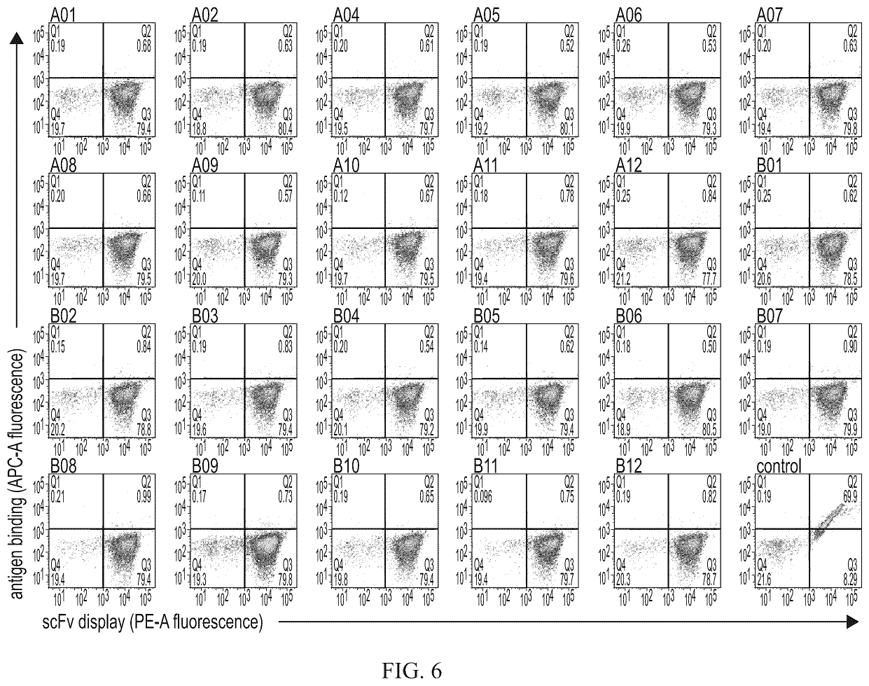

Of the identified clones, 23 were expressed as scFv-Fc in S. cerevisiae yeast strain YVH10 (ATCC MYA-4940): 11 from the Combo 1 library (A01-A06, B01-B06) and 12 from the Combo 2 library (A07-A12, B07-B12). These had 3 to 13 amino acid changes from the parental. To determine whether they retained the same epitope as the initial lead, it was tested if these were able to inhibit the parental scFv from binding to the antigen. The present disclosure incubated the scFv supernatants with the labeled antigen (10 nM) for 15 minutes. Approximately 2×10⁵ yeast cells displaying the parental molecule were added to the mixture and incubated for 30 min at room temperature. Cells were washed twice and stained to detect binding using anti-SV5 labeled with PE (phycoerythrin; labels cells displaying scFv) and streptavidin labeled with Alexa Fluor 633. Populations were analyzed by flow cytometry for binding. All 23 affinity matured clones abolished any detectable binding to the antigen by the parental, while a control scFv supernatant directed against an unrelated target did not (FIG. 5A and FIG. 6). While precise determination of the epitope recognized by all clones would require structural analysis, the fact that all these derive from the parental molecule, retain the same HCDR3 and framework regions, and compete for antigen binding suggests they indeed recognize the same epitope in the target molecule.

After the initial identification of leads binding to a target of interest, clones are often tested for their ability to achieve the desired biological activity. At this stage, the epitope recognized by the antibody will dictate if the antibody will be an agonist, antagonist, or have no activity whatsoever. After leads with desired activity are identified, it is assumed that increasing affinity will also increase potency (Rosenfeld et al. 2017; Hurlburt et al. 2020), and retaining the recognition site is one of the basic requirements of this assumption. More aggressive maturation techniques such as chain shuffling (especially heavy chain, or portions thereof) may have a higher potential to cause epitope drift and loss of activity, making a previously valuable molecule useless. The present disclosure has shown that a stepwise approach where at least 4 CDRs remain constant, and very importantly, the HCDR3 remains unchanged, was effective in obtaining very high affinities while retaining recognition of the same epitope. Whereas every antibody-antigen pair had its own peculiarities, it was believed that the centrality of the HCDR3 in epitope recognition (Xu and Davis 2000, Akbar, Robert et al. 2021) justified the retention of the parental HCDR3 to avoid epitope drift.

### Example 7: Affinity Screening of Affinity Matured Clones

Finally, the present disclosure determined the affinity of the same 23 clones using high-throughput SPR. The Carterra LSA surface resonance system was used for the affinity measurements. Briefly, anti-Human IgG Fe (Southern Biotech, #2048-01) was chemically coupled to an HC30M chip following manufactures protocols. Crude yeast supernatants containing the scFv-Fc fusions were arrayed on the chip. Non-biotinylated antigen was injected at varying concentrations (0.08 nM to 50 nM) to determine association and dissociation rates. All analyses were performed using Carterra software and sensorgrams were fitted using a pseudo first order kinetic model (Lundström 1994). All clones evaluated had a dissociation rate (kd) 10-200-fold slower than the parental antibody (FIGS. 5B-5C) whereas their association rates (ka) were basically unchanged. In fact, it is notable that the off rates for some of the tested antibodies are probably better than 10⁻⁵, i.e., 500-fold (FIGS. 5B-5C), as indicated by the bunching of matured antibodies at that value (FIG. 5C)—such slow off rates are challenging to accurately measure using this technique. No affinity difference was observed between clones coming from one combo library versus the other. The improvement of off-rates, but not on-rates, is a reflection of the protocols and selective pressure used during library panning: extended periods of antigen release favored stable binders with long off rates, while no effort was made to rescue clones that had faster association. Dissociation constants (KD) for the duplicates showed good agreement in absolute terms with the highest deviation being 0.18 nM in clone B09 (FIG. 5C).

This work demonstrates the possibility of performing affinity maturation of a low-nanomolar affinity antibody to the low-picomolar range by replacing all CDRs, except HCDR3, with a collection of known human CDRs. Replacing no more than two CDRs in phase 1 with compatible CDRs from similar antibodies, along with retention of the HCDR3, is expected to maintain epitope binding, given the importance of HCDR3 in antibody binding (Xu and Davis 2000). Affinity gain came mostly from improved off-rate (kd) which has been suggested to better correlate with the biological activity than the affinity itself (KD) (Rosenfeld et al. 2017; Hurlburt et al. 2020). Off rates for about half the antibodies were measured as <10⁻⁵, which is the measurement limit for SPR. It is possible that off rates are actually longer, and corresponding affinities higher, if measured using alternative methods, such as kinetic exclusion assays (Darling and Brault 2004). Although a set of CDRs were used in this work that came from an internal library, one could easily obtain sequences from publicly available datasets (Kovaltsuk, Leem et al. 2018). It is expected that each antibody-antigen pair will have its own peculiarities and tolerance for mutations at distinct positions. Nonetheless, the concepts and overall experimental design proposed here are expected to be equally useful for other antibodies as well based on the results disclosed herein.

### Example 8: Parental Antibody Identification and CDR Design Including HCDR3

In some cases, it may be desirable to introduce limited diversity into the HCDR3 as well. As the HCDR3 is considered the most important CDR mediating antibody binding specificity, extensive mutation in the HCDR3 may be detrimental, in that binding specificity of the antibody under examination may be modified. An antibody against a monomeric human protein of therapeutic interest was chosen to be affinity matured using the improved affinity maturation method. The antibody comprises a VH3 heavy chain and a VK1-39 light chain. Analysis of the CDRs revealed three sequence liabilities with the potential of affecting downstream clinical development: two distinct YY polyreactivity sites, which can lead to aggregation and non-specificity at HCDR3 and LCDR3, and one hydrophobic site at LCR2. The scFv affinity (K_{D}) was determined by yeast display flow cytometry. Briefly, cells were washed twice with cold washing buffer (PBS pH 7.4 0.5% BSA) and incubated at room temperature with different concentrations of biotinylated antigen, from 4 nM to 3 μM. After the biotinylated antigen incubation step the cells are washed and stained with the anti-SV5 labeled with PE (phycoerythrin; labels cells displaying scFv) and streptavidin labeled with Alexa Fluor 633 (Thermo Scientific; labels cells bound to biotinylated antigen) and then cells are analyzed by flow cytometry. Parental clone affinity was estimated at 413.7±33.27 nM (n=3).

To identify CDRs that could be shuffled into the parental sequence for affinity maturation, the present disclosure used deep sequencing data (MiSeq and NovaSeq) from a naïve semisynthetic library (Azevedo Reis Teixeira, Andre et al. 2021, Bradbury, A. R. M. et al. 2020). In this library, CDRs from B cell receptor antibodies are embedded within well-behaved clinical antibody scaffolds, the HCDR3s, due to their high diversity, are directly amplified from B cell mRNA, and the remaining naturally replicated CDRs are identified from next generation sequencing (NGS) of numerous donors and synthesized on arrays after eliminating previously identified sequence liabilities. For LCDR1-3 only CDR sequences coming from the VK1 germline family were considered and for HCDR1-2 only sequences coming from the VH3 family were considered, assuming that these sequences, since they belonged to the same germline genes as the parental antibody, would be better tolerated and minimize any potential structural disruption. To minimize the chance of selecting antibodies with unfavorable developability profiles, sequences containing undesirable liabilities such as glycosylation sites and unpaired cysteines were eliminated (see, Table 5 for the full list). Table 5 shows list of sequence liabilities removed from CDRs designed for affinity maturation using four Phase I libraries. Every CDR identified containing any of the listed motifs were discarded.

TABLE 5
List of Sequence Liabilities Removed from CDRs Designed
for Affinity Maturation Using Four Phase I Libraries

| Liability | Motifs | Reference |
|---|---|---|
| N-linked | NxS and NxT, where x | Gavel and von |
| glycosylation | is not a Pro | Heijne 1990; Kumar |
| | | and Singh 2016 |
| Asn deamidation | NG, NS, NT, NN, | Sydow, Lipsmeier |
| | GNF, GNY | et al. 2014; Kumar |
| | | and Singh 2016 |
| Asp isomerization | DG, DS, DD | Sydow, Lipsmeier |
| | | et al. 2014 |
| Polyreactivity | GG, RR, VG, VV, | Kelly, Le et |
| | YY, WW, WxW | al. 2018 |
| Aggregation | FHW | Wu, Luo et al. |
| | | 2010; Bethea, |
| | | Wu et al. 2012 |
| Cys | All Cys | Kumar and Singh |
| | | 2016 |
| Proteolysis | DP | Vlasak and |
| | | Ionescu 2011 |
| Glutamine | QG | |
| deamination | | |
| Charge | Net charge ≥+1 in | |
| | LCDR1-3, HCDR1-2 | |
| Hydrophobicity | Parker hydropathy | |
| | index <0 | |

For HCDR3, a mutational scanning approach was taken: each amino acid position was mutated to 19 amino acids (excluding cysteine) for a total of Y×19 oligonucleotides, where Y is the number of HCDR3 amino acids to be mutated. The present disclosure believe the centrality of the HCDR3 in epitope recognition (Xu and Davis 2000, Akbar, Robert et al. 2021) justifies the application of the mutational scanning approach to identify mutations that are better tolerated and to simultaneously avoid epitope drift, minimizing any potential structural disruption.

The identified CDRs were generated with flanking framework sequences matching the parental antibody and produced using array-based DNA synthesis (Agilent). This allowed the rescue of the full diversity at each individual CDR site by using framework primers.

### Example 9: Construction of Four Phase 1 Libraries

Library size is often a concern when performing in vitro evolution of any sort since one is limited by the number of transformants that can be conveniently obtained during library generation: 10⁹-10¹⁰ for phage and E. coli display and 10⁸-10⁹ for yeast display in S. cerevisiae. The present disclosure opted for a step-wise approach (Hemminki, Niemi et al. 1998) since it would allow exploring the sequence space more effectively: in phase 1, HCDR1 was combined with HCDR2, LCDR1 and LCDR2, and leave HCDR3 and LCDR3 by themselves (Table 6), but there was no reason to believe that this was somehow the optimal approach. It was done so out of what seemed to be the most convenient combinations for library building. These libraries allowed the present disclosure to generate more transformants than the maximum combinatorial diversities for each library (theoretical diversity ranging from 4.0×10⁴ to 2.8×10⁷). Also, by fixing at least four parental CDRs in each library, search space was decreased, increasing the chances of finding new variants binding to the antigen in the same way, essential to retaining biological activity. In addition to the incorporated CDR diversity, each library also included the parental CDR sequences at the same abundance as the other introduced CDRs, even if they contained sequence liabilities. This was to ensure retained activity in the case that particular parental CDR sequences were essential for binding. Table 6 shows number of different sequences introduced in each CDR position of light and heavy chain of four Phase I libraries. Theoretical diversity is calculated by the combinatorial potential of the CDRs and reported number of transformants correspond to the yeast display libraries created.

TABLE 6
Number of Different Sequences Introduced in Each CDR
Position of LC and HC of Four Phase I Libraries

| Family | Region | CDRs | Library | Diversity | Transformants |
|---|---|---|---|---|---|
| VH3 | HCDR1 | 4,545 | H1H2 | 2.8E+07 | 3.60E+08 |
| | HCDR2 | 6,099 | | | |
| | HCDR3 | 235 | H3 | 2.4E+02 | 1.60E+08 |
| Vk1 | LCDR1 | 3,694 | L1L2 | 1.7E+07 | 3.56E+08 |
| | LCDR2 | 4,707 | | | |
| | LCDR3 | 39,988 | L3 | 4.0E+04 | 1.42E+08 |

The six different collections of CDR sequences (HCDR1, HCDR2, HCDR3, LCDR1, LCDR2 and LCDR3) were amplified with specific primers by PCR using Q5 polymerase (NEB #M0491L). The remaining regions were amplified from the parental scFv and assembled with the CDRs by PCR (see, FIGS. 7A-7B). Libraries were assembled by combining the newly produced CDR pools with the remaining parts of the scFv. For example, the H1H2 library was assembled by (1) amplifying the HCDR1 and HCDR2 with the flanking frameworks from the synthetic oligo pool, (2) amplifying the remaining parts of the scFv from the parental clones, (3) assembling the produced fragments by overlap PCR, and (4) transforming the produced scFv cassettes into S. cerevisiae along with the digested yeast display vector (FIG. 7B). The scFv amplicons from each library were transformed into yeast along with the yeast display vector pSYD previously digested with the enzymes BssHII and NheI (NEB #R0199S and #R0131S) by electroporation using method described previously (Benatuil, Perez et al 2010).

The present disclosure chose to use scFv yeast display because it provides high precision in retrieving the desired population when combined with flow cytometry. However, it was expected this diversification approach was equally effective in the phage or ribosome display context, or any other display platform, and one could even expand possibilities due to larger library sizes. However, experimental design during selection would have to be adapted to these platforms since they are expected to show different behavior due to their monovalent nature as opposed to the multivalent nature of yeast: whereas a polyvalent yeast cell exists in a continuum from antigen saturation to no antigen binding that varies over time in proportion to the antibody off-rate, monovalent systems can only exist in the binary bound or not-bound states. This difference can be overcome, for example, by using a larger number of displaying particles and relying on the population binding decay over time as opposed to the single cell decay. The present disclosure routinely use the scFv format for antibody discovery and engineering and have found conversion to the IgG format occurs with 70-90% success. However, if conversion is a concern, the techniques described here would be easily applied using a Fab display system.

### Example 10: Antibody Selection by Yeast Display Using Four Phase 1 Libraries

Yeast display selections were performed as in Ferrara et al (2012). Briefly, cells were induced in selective media containing 2% galactose overnight at 20° C. 10⁵ induced cells are washed twice with cold washing buffer (PBS pH 7.4 0.5% BSA) and incubated at room temperature with the biotinylated antigen diluted in PBS. Equilibrium selection was performed by incubating the scFv-displaying yeast cells with decreasing concentrations of biotinylated antigen as the selections round progress and sorting labeled cells immediately after reaching equilibrium (FIG. 2A). After the biotinylated antigen incubation step the cells are washed and stained promptly with the anti-SV5 labeled with PE (phycoerythrin; labels cells displaying scFv) and streptavidin labeled with Alexa Fluor 633 (Thermo Scientific; labels cells bound to biotinylated antigen) and then cells binding the antigen are sorted either by FACS (fluorescence-activated cell sorting) or MACS (magnetic-activated cell sorting) (FIG. 2A). For the MACS procedure, paramagnetic beads coated with streptavidin were used (Miltenyi Biotec).

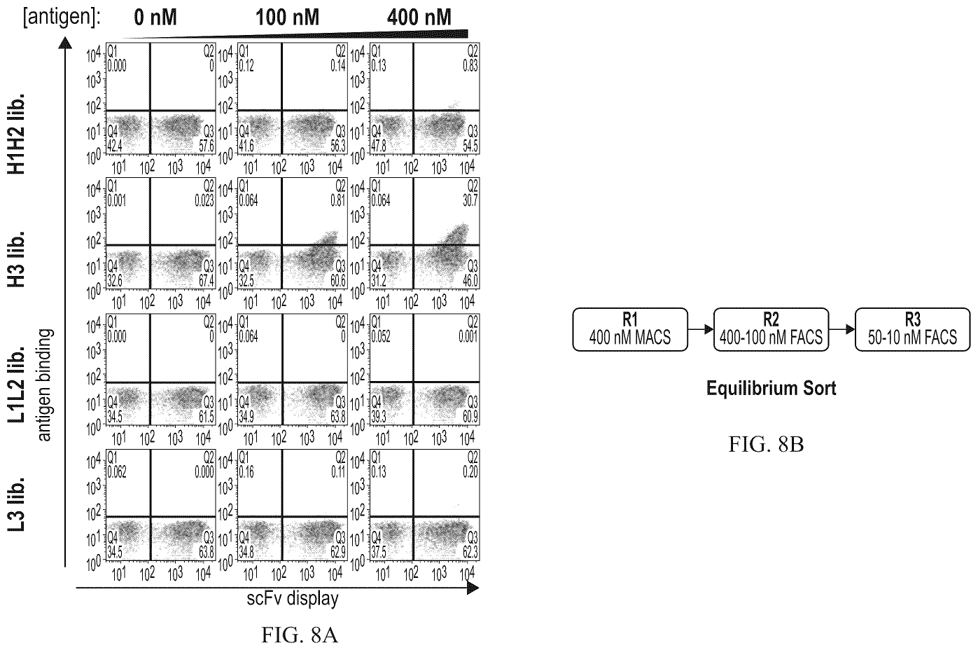

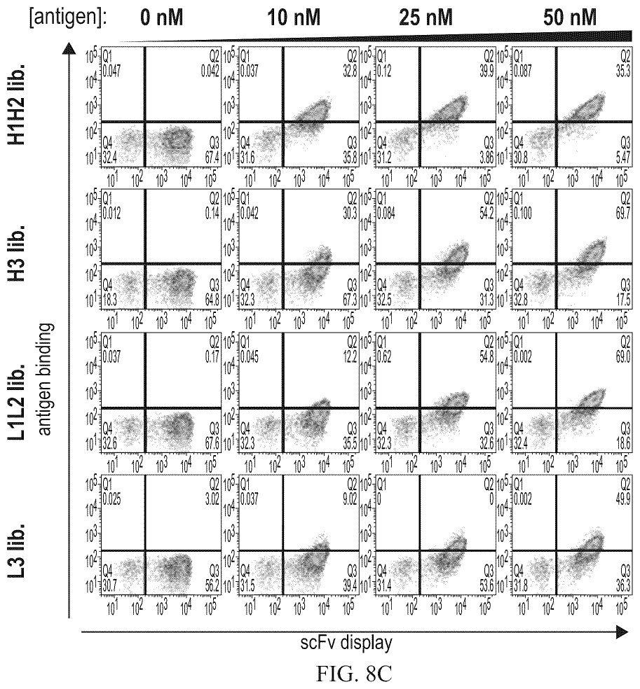

The present disclosure performed an initial flow cytometric assessment of the libraries using decreasing antigen concentrations (FIG. 8A). The light chain library L1L2 showed a reduced population binding to the antigen even at the highest concentration used: at 400 nM binding population of 0.031%. Libraries H1H2 and L3, showed a small population binding to the antigen at 400 nM: binding populations of 0.63% and 0.2% respectively. As for the heavy chain library H3, a significant population of 6.81% binding to the antigen even at 100 nM was identified, suggesting higher improvement potential for the heavy chain CDRs as opposed to the light chains for this particular clone.

Given the size of each of these libraries, the present disclosure performed the first round of selection using magnetic-assisted cell sorting (MACS) at antigen concentration of 400 nM (FIG. 8B). This allowed the present disclosure to label and sort a larger number of cells than what would be practical using a flow cytometer. For the two subsequent rounds, the present disclosure used fluorescence activated cell sorting (FACS) to enable more precise sorting of the cells of interest (FIG. 8B). An assessment of the population obtained for each of these libraries after three rounds show that in all cases a significant improvement of affinity can be observed (FIG. 8C): all generated populations show significant binding to the antigen even at 10 nM, a concentration at which the parental antibody does not bind. Even though affinities of individual clones have not been assessed at this stage, it is not unreasonable to assume that, given the yeast display staining profile, one could already find binders with satisfactory affinities depending on the requirements of the project.

### Example 11: Phase 2 Combo Libraries Construction from Four Phase 1 Libraries

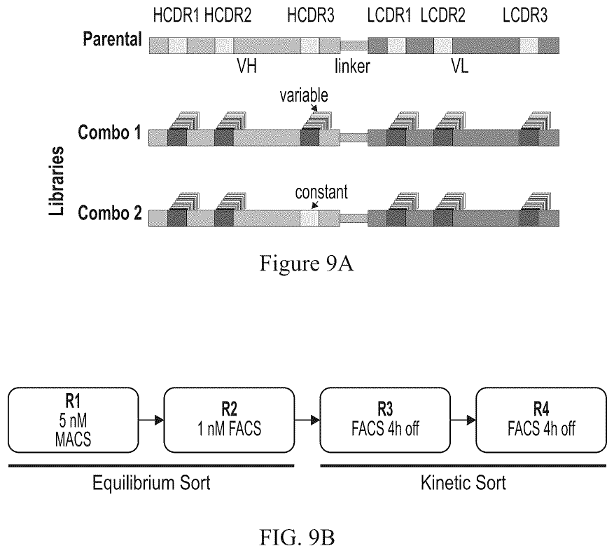

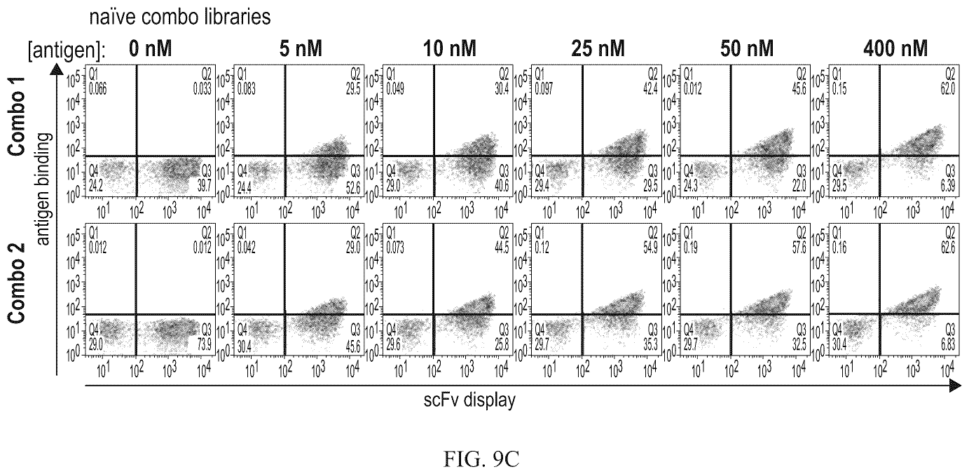

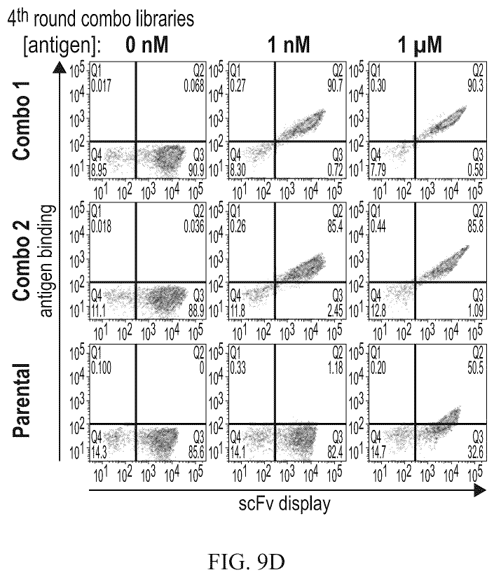

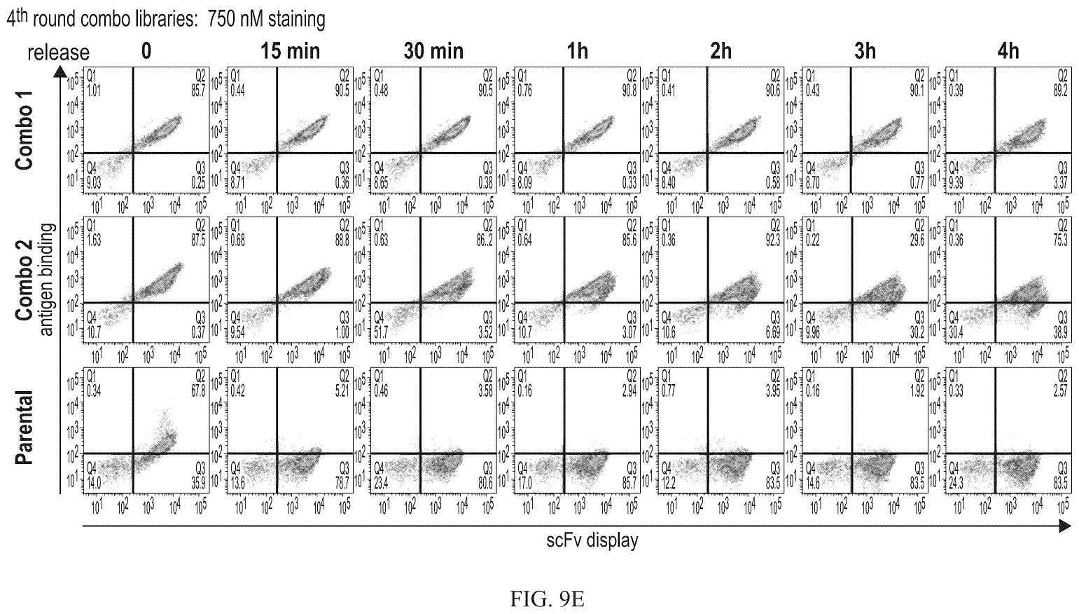

After selecting populations of CDRs at each position in the four initial libraries that showed improved binding, the present disclosure combined the selection outputs with the goal to further improve binding towards still slower off-rates. Two “combo” libraries were assembled by PCR: the first (Combo 1—6.8E+08 transformants) was created by combining the output of all four libraries (H1H2, H3, L1L2 and L3), while the second (Combo 2—6.5E+08 transformants) omitted the H3 output and used the parental HCDR3 (FIG. 3A and FIG. 9A). Two different selection strategies were used in this phase: equilibrium selection, the more traditional approach used in phase I, and kinetic selection (FIG. 9B) (Boder and Wittrup 1998, Boder, Midelfort et al. 2000). Equilibrium selection was performed by incubating the scFv-displaying yeast cells with decreasing concentrations of biotinylated antigen as the selections round progress. Decreasing the antigen concentration cannot be carried out indefinitely since the displayed antibodies on the yeast surface will deplete antigen from the solution before reaching equilibrium (VanAntwerp and Wittrup 2000). Avoiding this requires the use of large and impractical incubation volumes with small numbers of cells, to ensure minimal antigen is removed and the effective concentration remains constant. An alternative to equilibrium selection is kinetic selection: scFv-displaying yeast cells are incubated with the labeled antigen, washed, incubated with unlabeled antigen (10× more concentrated than the biotinylated antigen) to select only clones with stable binding to the antigen (slow off-rate—k_{d}). The unlabeled antigen is used to prevent rebinding of the displaced labeled antigen. After a defined period, cells still bound to the labeled antigen were stained and sorted as described before.

An approach combining equilibrium and kinetic sorts was used (FIGS. 2A and 9C) with 2 successive rounds of equilibrium sorting at 5 nM and 1 nM of antigen concentration respectively, followed by two rounds of kinetic sorting at 4 h with unlabeled antigen. An initial assessment showed that straight after transformation the libraries were already showing binding to the antigen even at 5 nM—19.0% and 29.0% of the population for Combo 1 and Combo 2 libraries, respectively (FIG. 9C). After four rounds of sorting, the difference between the combo libraries and the parental antibody is striking: at 1 nM>90% and >85% of the yeast population is bound to the antigen for the Combo 1 and 2 libraries respectively, as opposed to 1.18% for the parental antibody (FIG. 9D). When stained with 750 nM of antigen and destained for 4 h, the combo libraries show very little signal loss compared to the staining at 750 nM of antigen with no destaining period, whereas the parental completely lost binding under these conditions, demonstrating significant improvement of off-rate (kd) for the selected Combo libraries (FIG. 9E).

### Example 12: Sanger Sequence Screening of Affinity Matured Clones from Four Phase I Libraries

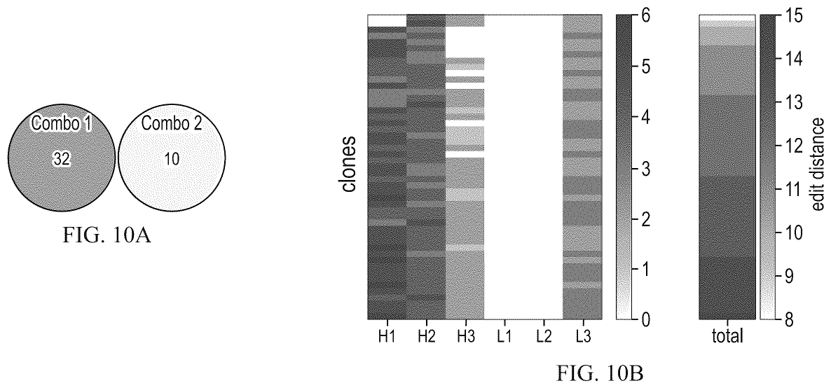

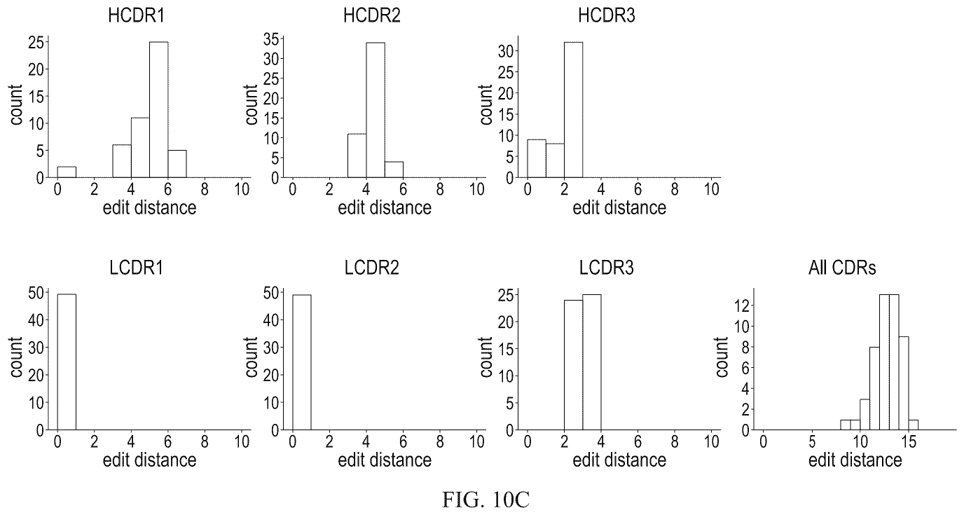

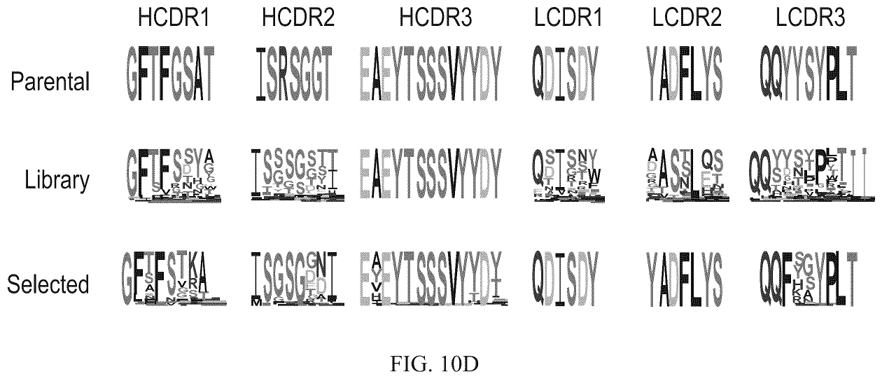

To evaluate the clones obtained the present disclosure submitted 60 colonies from the final rounds of selection of each Combo library for Sanger sequencing. Fifty-two (52) clones were sequenced, and 51 unique sequences were identified, with no overlap between libraries (39 unique clones from Combo 1, and 21 unique clones from Combo 2 (FIG. 10A). Interestingly, the heavy chain CDRs were more diverse than the light CDRs (Table 7)—this relates to the observed binding pattern of the first naïve libraries that showed higher binding signal for H1H2 and H3 libraries (FIG. 8A) suggesting that the heavy CDRs indeed were more tolerant to sequence changes, and hence could have a greater contribution to improving antibody affinity.

TABLE 7
Number of Unique CDRs Found in 52 Sequenced Clones

| Region | Combo 1 | Combo 2 |
|---|---|---|
| HCDR1 | 22 | 7 |
| HCDR2 | 25 | 7 |
| HCDR3 | 21 | 1 |
| LCDR1 | 1 | 1 |
| LCDR2 | 1 | 1 |
| LCDR3 | 16 | 8 |

The minimal number of CDR mutations in the antibodies was 8 total changes, and up to 15 amino acid changes (FIG. 10B). The most frequent number of mutations was 12 (FIGS. 10B and C), a caveat here being that some of the clones came from the Combo 2 library in which HCDR3 was kept constant. For HCDR1, the dominant sequence had 5 mutations from parental, and for HCDR2 4 mutations was the most frequently observed change (FIG. 10C). For Combo library 1 HCDR3, 2 mutations or less from the parental were the most frequently observed changes (FIG. 10C). Although library design only included one mutation in Combo 1 HCDR3, an additional mutation was likely generated and favored during the selection of high-affinity clones. No change to the parental sequence was observed for LCDR1 and LCDR2, regardless of the Combo library design (FIG. 9B). For LCDR3, the dominant sequence had 3 mutations from parental (FIG. 10C).

Sequence comparison of the CDRs present in the parental antibody, the maturation libraries, and the identified clones (FIG. 10D) shows that in many cases the amino acid in the parental antibody was optimal since after several rounds of selections the clones converge back to the same sequence. While there was high tolerance for diversity overall, LCDR1 and LCDR2 do not tolerate changes at all and all clones converge back to the parental sequence, justifying the inclusion of the parental CDR sequences in the libraries. Also, much of the diversity found is concentrated in a few positions/hotspots, suggesting that these may be less relevant for binding (e.g.: position 4 and 5 at LCDR3, FIG. 10D), while some other positions showed clear convergence to an amino acid different to the parental (e.g.: position 3 (Y to F) at LCDR3, FIG. 10D).

None of the affinity matured antibodies had sequence liabilities in HCDR1, HCDR2, LCDR1 and LCDR3. Since all the identified clones converged back to the parental LCDR2 sequence, the hydrophobic site at LCR2 was maintained. HCDR3 remained unchanged from the parental in Combo library 2, and clones derived from that library retained the YY polyreactivity site. As for Combo library 1, identified clones had high tolerance for diversity on HCDR3, however, most clones retained the second YY polyreactivity site and were mutated at a different position. Nevertheless, 11 clones derived from Combo library 1 were free of liabilities at HCDR3. In summary, 11 affinity matured clones retained a single CDR liability (hydrophobic site at LCR2), 40 clones had two sequence liabilities (hydrophobic site at LCR2 and YY polyreactivity site at HCDR3), while the parental clone had 3 sequence liabilities, showing that the method can be effectively used to reduce developability issues simultaneously with affinity maturation.

In theory, one could omit original parental CDRs containing sequence liabilities in the affinity maturation libraries, but at the risk of being unable to mature the clone since the eliminated motif may be essential for binding, as seems to be the case for the hydrophobic motif in the LCDR2 of this clone. In the affinity maturation of this antibody, the present disclosure observed that HCDR1, HCDR2, HCDR3 and LCDR3 were much more amenable to substitutions than LCDR1 and LCDR2. This was reflected in the naïve L1L2 library showing significantly less binding at the beginning of the process (FIG. 8A). Hypothesizing that the lack of improvement with the L1L2 library may reflect the presence of optimal sequences for these CDRs. In fact, all the unique clones identified from the combo libraries had unmutated parental LCDR1-2, confirming the importance of these CDRs in binding.

### Example 13: Screening of Affinity Matured Clones from Four Phase I Libraries

To evaluate monoclonal affinity, 12 unique clones from the final round of selection of Combo 1 and Combo 2 libraries were randomly selected for monoclonal binding verification as yeast display by flow cytometry. Cells were washed twice with cold washing buffer (PBS pH 7.4 0.5% BSA) and incubated at room temperature with different concentrations of biotinylated antigen, from 500 down to 2 nM. After the biotinylated antigen incubation step the cells are washed and stained with the anti-SV5 labeled with PE (phycoerythrin; labels cells displaying scFv) and streptavidin labeled with Alexa Fluor 633 (Thermo Scientific; labels cells bound to biotinylated antigen) and then cells are analyzed by flow cytometry. The mean affinity for clones selected from Combo 1 library was 5.609±0.4931 nM, ranging from 3.879 to 9.927 nM. As for the 12 clones selected from Combo 2 library, mean affinity was 8.075±1.758 nM, ranging from 4.215 to 24.84 nM (Table 8). Compared to the parental clone, which has an affinity of 413.7±33.27 nM measured by yeast display. 3 affinity matured clones from the Combo 1 library had ≥100-fold improvement in affinity, while 2 affinity matured clones from Combo 2 library had ≥90-fold improvement in affinity (Table 8). The performances of both Combo libraries were equivalent during monoclonal affinity screening (one-way ANOVA with Tukey's multiple comparisons test: not statistically significant), however, mean affinities from both Combo libraries were significantly different from the parental (one-way ANOVA with Tukey's multiple comparisons test: Combo 1 P≤0.01, Combo 2 P≤0.05).

TABLE 8
Kinetic Measurements as Yeast-Display for
Affinity Matured Clones and Parental

| | | K_{D} | R | Fold |
|---|---|---|---|---|
| Library | Clone | (nM) | squared | improvement |
| Combo 1 | A02 | 3.879 | 0.9967 | 106.7 |
| | A05 | 4.108 | 0.9969 | 100.7 |
| | A09 | 4.111 | 0.9950 | 100.6 |
| | A06 | 4.295 | 0.9966 | 96.3 |
| | A12 | 4.998 | 0.9958 | 82.8 |
| | A01 | 5.173 | 0.9954 | 80.0 |
| | A03 | 5.362 | 0.9965 | 77.2 |
| | A04 | 5.495 | 0.9973 | 75.3 |
| | A11 | 6.24 | 0.9985 | 66.3 |
| | A08 | 6.702 | 0.9966 | 61.7 |
| | A10 | 7.023 | 0.9930 | 58.9 |
| | A07 | 9.927 | 0.9945 | 41.7 |
| Combo 2 | A07 | 4.215 | 0.9957 | 98.1 |
| | A05 | 4.407 | 0.9954 | 93.9 |
| | A12 | 4.708 | 0.9917 | 87.9 |
| | A03 | 5.133 | 0.9900 | 80.6 |
| | A08 | 5.478 | 0.9937 | 75.5 |
| | A04 | 5.489 | 0.9916 | 75.4 |
| | A06 | 5.912 | 0.9998 | 70.0 |
| | A10 | 6.072 | 0.9892 | 68.1 |
| | A11 | 7.288 | 0.9997 | 56.8 |
| | A02 | 7.808 | 0.9955 | 53.0 |
| | A01 | 15.55 | 0.9971 | 26.6 |
| | A09 | 24.84 | 0.9978 | 16.7 |

| | | | |
|---|---|---|---|
| Parental | 413.7 | 0.9907 | 1 |

This work demonstrates the possibility of performing affinity maturation of an antibody by replacing all CDRs with a collection of known human CDRs, while mutationally scanning HCDR3. Replacing no more than two CDRs in phase 1 with compatible CDRs from similar antibodies, along with only point mutation variation of HCDR3, is expected to maintain epitope binding, given the importance of HCDR3 in antibody binding (Xu and Davis 2000). Improved off-rate (kd), which has been suggested to better correlate with the biological activity than the affinity itself (KD), was demonstrated for both Combo libraries (Rosenfeld et al. 2017; Hurlburt et al. 2020). Although a set of CDRs was used in this work that came from an internal library, one could easily obtain sequences from publicly available datasets (Kovaltsuk, Leem et al. 2018). It is expected that each antibody-antigen pair will have its own peculiarities and tolerance for mutations at distinct positions in different CDRs. Nonetheless, the concepts and overall experimental design proposed here are expected to be equally useful for other antibodies as well based on the results disclosed herein.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An antibody library comprising a plurality of antibodies, wherein each antibody of the plurality of antibodies comprises the following CDR sequences:
a V_{H}-CDR1 sequence, a V_{H}-CDR2 sequence, a V_{H}-CDR3 sequence,
a V_{L}-CDR1 sequence, a V_{L}-CDR2 sequence, and a V_{L}-CDR3 sequence,
wherein two or more of the CDR sequences are the same for each antibody of the plurality of antibodies (invariant CDR sequences), wherein the two or more invariant CDR sequences are derived from a parental antibody to be affinity matured; and
wherein each of the remaining CDR sequences comprises a plurality of CDRs derived from the sequences of CDRs found in naturally occurring antibodies (variant CDR sequences).

2. The antibody library of claim 1, wherein the two or more invariant CDR sequences are selected from the group consisting of:
(i) V_{L}-CDR3, V_{H}-CDR1, V_{H}-CDR2, and V_{H}-CDR3;
(ii) V_{L}-CDR1, V_{L}-CDR2, V_{H}-CDR1, V_{H}-CDR2, and V_{H}-CDR3;
(iii) V_{L}-CDR1, V_{L}-CDR2, V_{L}-CDR3, and V_{H}-CDR3;
(iv) V_{L}-CDR1, V_{L}-CDR2, and V_{H}-CDR3; and
(v) V_{L}-CDR1, V_{L}-CDR2, V_{L}-CDR3, V_{H}-CDR1 and V_{H}-CDR2.

3. An antibody library comprising a plurality of antibodies, wherein each antibody of the plurality of antibodies comprises the following CDR sequences:
a V_{H}-CDR1 sequence, a V_{H}-CDR2 sequence, a V_{H}-CDR3 sequence,
a V_{L}-CDR1 sequence, a V_{L}-CDR2 sequence, and a V_{L}-CDR3 sequence,
wherein two or more of the CDR sequences are the same for each antibody of the plurality of antibodies (invariant CDR sequences), wherein the two or more invariant CDR sequences are derived from a parental antibody to be affinity matured; and
wherein each of the remaining CDR sequences (variant CDR sequences), except the HCDR3, comprises a plurality of CDRs derived from the sequences of CDRs found in naturally occurring antibodies, and the HCDR3 comprises a plurality of variants of the parental HCDR3.

4. The antibody library of claim 3, wherein the two or more invariant CDR sequences are selected from the group consisting of:
(i) V_{L}-CDR3, V_{H}-CDR1, V_{H}-CDR2, and V_{H}-CDR3;
(ii) V_{L}-CDR1, V_{L}-CDR2, V_{H}-CDR1, V_{H}-CDR2, and V_{H}-CDR3;
(iii) V_{L}-CDR1, V_{L}-CDR2, V_{L}-CDR3, and V_{H}-CDR3;
(iv) V_{L}-CDR1, V_{L}-CDR2, and V_{H}-CDR3; and
(v) V_{L}-CDR1, V_{L}-CDR2, V_{L}-CDR3, V_{H}-CDR1, and V_{H}-CDR2.

5. The antibody library of claim 3, wherein an antibody selected from the library binds more tightly to a target antigen or a target antigenic epitope relative to a parental antibody.

6. An antibody library that comprises a plurality of antibodies, wherein each antibody of the plurality of antibodies comprises:
a V_{H}-CDR1 sequence, a V_{H}-CDR2 sequence, a V_{H}-CDR3 sequence, and
a V_{L}-CDR1 sequence, a V_{L}-CDR2 sequence, and a V_{L}-CDR3 sequence, wherein the CDR sequences of the antibody library are derived from the combination of the CDR pluralities selected in claim 3.

7. The antibody library of claim 5, wherein the HCDR3 comprises single mutations of the parental HCDR3.

8. The antibody library of claim 1, wherein the invariant CDR sequences comprise a combination of V_{H}-CDR1, V_{H}-CDR2, V_{H}-CDR3, V_{L}-CDR1, V_{L}-CDR2 and/or V_{L}-CDR3.

9. The antibody library of claim 1, wherein the variant CDR sequences are free of one or more of the following liabilities:
(i) a glycosylation site;
(ii) a deamidation site;
(iii) an isomerization site;
(iv) unpaired cysteine;
(v) net charge greater than 1;
(vi) a tripeptide motif containing at least two aromatic residues;
(vii) a motif that promotes aggregation;
(viii) a poly specificity site;
(ix) a protease sensitive site;
(x) an integrin binding site;
(xi) a lysine glycosylation site;
(xii) a metal catalyzed fragmentation site;
(xiii) a poly specificity aggregation site; and
(xiv) a streptavidin binding motif.

10. The antibody library of claim 9, wherein the glycosylation site comprises the motif NXS, NXT, or NXC, in which X represents any naturally-occurring amino acid residue except for proline; wherein the deamidation site comprises the motif of NG, NS, NT, NN, NA, NH, ND, GNF, GNY, GNT, or GNG; wherein the isomerization site comprises the motif of DT, DH, DS, DG, or DD; wherein the tripeptide is HYF or HWH; wherein the motif that promotes aggregation comprises the motif of FHW; wherein the poly specificity site comprises the motif of GG, GGG, RR, VG, W, WV, WW, WWW, YY, or WXW, in which X represents any amino acid residue; wherein the protease cleavage site comprises the motif of DX, in which X is P, G, S, V, Y, F, Q, K, L, or D; wherein the integrin binding site comprises RGD, RYD, LDV, or KGD; wherein the lysine glycosylation site comprises KE, EK, or ED; wherein the metal catalyzed fragmentation site comprises the motif of HS, SH, KT, HXS, or SXH, in which X represents any amino acid residue; wherein the poly specificity aggregation site comprises a motif of X1X2X3, wherein each of Xi, X2, and X3 independently is selected from the group consisting of F, I, L, V, W and Y; and/or wherein the streptavidin binding motif comprises the motif HPQ, EPDW, PWXWL, in which X represents any amino acid residue, GDWVFI, or PWPWLG.

11. The antibody library of claim 1, wherein the antibody library is a full-length antibody library, a Fab antibody library, a single-chain antibody library, or a single domain antibody library.

12. The antibody library of claim 1, wherein the antibody library is a human antibody library.

13. A method for generating an antibody library, the method comprising:
(a) selecting two or more CDR sequences from a parental antibody to be affinity matured, wherein the two or more CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence; and
(b) generating an antibody library comprising a plurality of antibodies, wherein each antibody of the plurality of antibodies comprises the following CDR sequences:
(i) the two or more CDR sequences selected in (a) (invariant CDR sequences); and
(ii) a unique combination of remaining CDR sequences not selected in (a) (variant CDR sequences), wherein the variant CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence and are derived from the sequences of CDRs found in naturally occurring antibodies.

14. A method for generating an antibody library, the method comprising:
(a) selecting two or more CDR sequences from a parental antibody to be affinity matured, wherein the two or more CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence; and
(b) generating an antibody library comprising a plurality of antibodies, wherein each antibody of the plurality of antibodies comprises the following CDR sequences:
(i) the two or more CDR sequences selected in (a) (invariant CDR sequences); and
(ii) a unique combination of remaining CDR sequences not selected in (a) (variant CDR sequences), wherein the variant CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence and are derived from the sequences of CDRs found in naturally occurring antibodies for all CDR sequences except V_{H}-CDR3 (HCDR3), wherein the HCDR3 comprises a plurality of variants of the parental HCDR3.

15. The method of claim 14, wherein the HCDR3 comprises single mutation(s) of the parental HCDR3 sequence.

16. A method for generating an antibody library, the method comprising:
(a) selecting two or more CDR sequences from a parental antibody to be affinity matured, wherein the two or more CDR sequences are selected from the group consisting of a V_{H}-CDR1 sequence, a V_{H}-CDR2 sequence, a V_{H}-CDR3 sequence, a V_{L}-CDR1 sequence, a V_{L}-CDR2 sequence, and a V_{L}-CDR3 sequence; and
(b) generating an antibody library comprising a plurality of antibodies, wherein each antibody of the plurality of antibodies comprises the following CDR sequences:
(i) the two or more CDR sequences selected in (a) (invariant CDR sequences), wherein the CDR sequences of the antibody library are derived from the combination of the CDR pluralities selected from the group consisting of:
[a] V_{L}-CDR3, V_{H}-CDR1, V_{H}-CDR2, and V_{H}-CDR3;
[b] V_{L}-CDR1, V_{L}-CDR2, V_{H}-CDR1, V_{H}-CDR2, and V_{H}-CDR3;
[c] V_{L}-CDR1, V_{L}-CDR2, V_{L}-CDR3, and V_{H}-CDR3;
[d] V_{L}-CDR1, V_{L}-CDR2, and V_{H}-CDR3; and
[e] V_{L}-CDR1, V_{L}-CDR2, V_{L}-CDR3, V_{H}-CDR1, and V_{H}-CDR2; and
(ii) a unique combination of remaining CDR sequences not selected in (a) (variant CDR sequences).

17. A method for identifying an antibody with improved affinity to a target antigen or a target antigenic epitope, the method comprising:
(a generating an antibody library according to the method of claim 13; and
(b) screening the antibody library to isolate an antibody that binds more tightly to the target antigen or target antigenic epitope relative to a control, thereby identifying an antibody with improved affinity.

18. The method of claim 17, further comprising the additional step of:
(a) generating an antibody library wherein the CDR sequences of the antibody library are derived from the combination of the CDR pluralities selected in claim 16; and
(b) screening the antibody library to isolate an antibody that binds more tightly to the target antigen or target antigenic epitope relative to the parental antibody, thereby identifying an antibody with improved affinity.

19. A method to improve the developability of an antibody, the method comprising:
(a) generating an antibody library according to the method of claim 13, and wherein the invariant and variant CDR sequences are free of one or more of sequence liabilities; and
(b) screening the antibody library to isolate an antibody that binds more tightly to a target antigen or a target antigenic epitope relative to a control and lacks one or more of the sequence liabilities found in the CDRs of the original antibody, thereby improving the developability of the antibody.

20. The method of claim 17, wherein the control is a parental antibody.

21. The method of claim 13, wherein the two or more invariant CDR sequences are selected from the group consisting of:
(i) V_{L}-CDR3, V_{H}-CDR1, V_{H}-CDR2, and V_{H}-CDR3;
(ii) V_{L}-CDR1, V_{L}-CDR2, V_{H}-CDR1, V_{H}-CDR2, and V_{H}-CDR3;
(iii) V_{L}-CDR1, V_{L}-CDR2, V_{L}-CDR3, and V_{H}-CDR3;
(iv) V_{L}-CDR1, V_{L}-CDR2, and V_{H}-CDR3; and
(v) V_{L}-CDR1, V_{L}-CDR2, V_{L}-CDR3, V_{H}-CDR1 and V_{H}-CDR2.

22. The method of claim 13, wherein the invariant and variant CDR sequences are free of one or more of the following liabilities:
(i) a glycosylation site;
(ii) a deamidation site;
(iii) an isomerization site;
(iv) unpaired cysteine;
(v) net charge greater than 1;
(vi) a tripeptide motif containing at least two aromatic residues;
(vii) a motif that promotes aggregation;
(viii) a poly specificity site
(ix) a protease sensitive site;
(x) an integrin binding site;
(xi) a lysine glycosylation site;
(xii) a metal catalyzed fragmentation site;
(xiii) a poly specificity aggregation site; and
(xiv) a streptavidin binding motif.

23. The method of claim 22, wherein the glycosylation site comprises the motif NXS, NXT, or NXC, in which X represents any naturally-occurring amino acid residue except for proline; wherein the deamidation site comprises the motif of NG, NS, NT, NN, NA, NH, ND, GNF, GNY, GNT, or GNG; wherein the isomerization site comprises the motif of DT, DH, DS, DG, or DD; wherein the tripeptide is HYF or HWH; wherein the motif that promotes aggregation comprises the motif of FHW; wherein the poly specificity site comprises the motif of GG, GGG, RR, VG, W, WV, WW, WWW, YY, or WXW, in which X represents any amino acid residue; wherein the protease cleavage site comprises the motif of DX, in which X is P, G, S, V, Y, F, Q, K, L, or D; wherein the integrin binding site comprises RGD, RYD, LDV, or KGD; wherein the lysine glycosylation site comprises KE, EK, or ED; wherein the metal catalyzed fragmentation site comprises the motif of HS, SH, KT, HXS, or SXH, in which X represents any amino acid residue; wherein the poly specificity aggregation site comprises a motif of X1X2X3, wherein each of Xi, X2, and X3 independently is selected from the group consisting of F, I, L, V, W and Y; and/or wherein the streptavidin binding motif comprises the motif HPQ, EPDW, PWXWL, in which X represents any amino acid residue, GDWVFI, or PWPWLG.

24. The method of claim 13, wherein the antibody library is a full-length antibody library, a Fab antibody library, a single-chain antibody library, or a single domain antibody library.

25. The method of claim 13, wherein the antibody library is a human antibody library.

26. An antibody obtained according to the method of claim 17.

27. A method for generating a combinatorial antibody library, comprising
(a) obtaining two or more antibody libraries generated according to the method of claim 13;
(b) combining the two or more antibody libraries obtained in step (a) to form a combinatorial antibody library.

28. A VHH library comprising a plurality of antibodies, wherein each antibody of the plurality of antibodies comprises the following CDR sequences:
a CDR1 sequence, a CDR2 sequence, and a CDR3 sequence,
wherein one or more of the CDR1 sequence, CDR2 sequence, or CDR3 sequence are the same for each antibody of the plurality of antibodies (invariant CDR sequence(s)), wherein the one or more CDR1 sequence, CDR2 sequence, or CDR3 sequence are derived from a parental antibody to be affinity matured; and
wherein each of the remaining CDRs for each antibody of the plurality of antibodies comprises a plurality of CDR sequences derived from the sequences of CDRs found in naturally occurring antibodies (variant CDRs).

29. The VHH library of claim 28, wherein each antibody in the library binds more tightly to a target antigen or a target antigenic epitope relative to a control.

30. A VHH library comprising a plurality of antibodies, wherein each antibody of the plurality of antibodies comprises the following CDR sequences:
a CDR1 sequence, a CDR2 sequence, and a CDR3 sequence,
wherein one or more of the CDR1 sequence, CDR2 sequence, or CDR3 sequence are the same for each antibody of the plurality of antibodies (invariant CDR sequence(s)), wherein the one or more CDR1 sequence, CDR2 sequence, or CDR3 sequence are derived from a parental antibody to be affinity matured; and
wherein each of the remaining CDR sequences, except the HCDR3 sequence, comprises a plurality of CDR sequences derived from the sequences of CDRs found in naturally occurring antibodies (variant CDRs), and wherein the HCDR3 sequence comprises a plurality of variants of the parental HCDR3 sequence.

31. The VHH library of claim 30, an antibody selected from the library binds more tightly to a target antigen or a target antigenic epitope relative to a parental antibody.

32. The VHH library of claim 30, wherein the HCDR3 sequence comprises single mutations of the parental HCDR3 sequence.

33. A method for generating a VHH library, the method comprising:
(a) selecting one or more CDR sequences from the group consisting of a CDR1 sequence, a CDR2 sequence, and a CDR3 sequence; and
(b) generating an antibody library comprising a plurality of antibodies, wherein each antibody of the plurality of antibodies comprises the following CDR sequences:
(i) the one or more CDR sequences selected in (a) (invariant CDR sequence(s)); and
(ii) a unique combination of remaining CDR sequences not selected in (a) (variant CDR sequences), wherein each of the variant CDR sequences comprises a plurality of CDR sequences derived from the sequences of CDR sequences found in naturally occurring antibodies.

34. A method for generating a VHH library, the method comprising:
(a) selecting one or more CDR sequences from the group consisting of a CDR1 sequence, a CDR2 sequence, and a CDR3 sequence; and
(b) generating an antibody library comprising a plurality of antibodies, wherein each antibody of the plurality of antibodies comprises the following CDR sequences:
(i) the one or more CDR sequences selected in (a) (invariant CDR sequence(s)); and
(ii) a unique combination of remaining CDR sequences not selected in (a) (variant CDR sequences), wherein each of the variant CDR sequences, except the HCDR3 sequence, comprise a plurality of CDR sequences derived from the sequences of CDR sequences found in naturally occurring antibodies, and wherein the HCDR3 sequence comprises a plurality of variants of the parental HCDR3 sequence.

35. The method of claim 34, wherein the CDR3 sequence comprises single mutations of the parental HCDR3 sequence.

36. The method of claim 33, wherein the invariant and variant CDR sequences are free of one or more of the following liabilities:
(i) a glycosylation site;
(ii) a deamidation site;
(iii) an isomerization site;
(iv) unpaired cysteine;
(v) net charge greater than 1;
(vi) a tripeptide motif containing at least two aromatic residues;
(vii) a motif that promotes aggregation;
(viii) a poly specificity site
(ix) a protease sensitive site;
(x) an integrin binding site;
(xi) a lysine glycosylation site;
(xii) a metal catalyzed fragmentation site;
(xiii) a poly specificity aggregation site; and
(xiv) a streptavidin binding motif.

37. The method of claim 36, wherein the glycosylation site comprises the motif NXS, NXT, or NXC, in which X represents any naturally-occurring amino acid residue except for proline; wherein the deamidation site comprises the motif of NG, NS, NT, NN, NA, NH, ND, GNF, GNY, GNT, or GNG; wherein the isomerization site comprises the motif of DT, DH, DS, DG, or DD; wherein the tripeptide is HYF or HWH; wherein the motif that promotes aggregation comprises the motif of FHW; wherein the poly specificity site comprises the motif of GG, GGG, RR, VG, W, WV, WW, WWW, YY, or WXW, in which X represents any amino acid residue; wherein the protease cleavage site comprises the motif of DX, in which X is P, G, S, V, Y, F, Q, K, L, or D; wherein the integrin binding site comprises RGD, RYD, LDV, or KGD; wherein the lysine glycosylation site comprises KE, EK, or ED; wherein the metal catalyzed fragmentation site comprises the motif of HS, SH, KT, HXS, or SXH, in which X represents any amino acid residue; wherein the poly specificity aggregation site comprises a motif of X1X2X3, wherein each of Xi, X2, and X3 independently is selected from the group consisting of F, I, L, V, W and Y; and/or wherein the streptavidin binding motif comprises the motif HPQ, EPDW, PWXWL, in which X represents any amino acid residue, GDWVFI, or PWPWLG.
